(19) United States
(12) Patent Application Publication
(10) Pub. No.: US 2026/0325600 A1
(43) Pub. Date: Oct. 22, 2026

(54) FORMULATIONS FOR INTRAOCULAR DELIVERY OF PEPTIDES DERIVED FROM TYPE IV COLLAGEN

(71) Applicant: ASCLEPIX THERAPEUTICS, INC., Baltimore, MD (US)

(72) Inventors: NIRANJAN BABU PANDEY, Baltimore, MD (US); Adam Christopher MIRANDO, Baltimore, MD (US); Thomas John ROBINSON, Baltimore, MD (US)

(21) Appl. No.: 19/105,910

(22) PCT Filed: Aug. 25, 2023
(86) PCT No.: PCT/US2023/072905

§ 371 Date: Feb. 24, 2025

Related U.S. Application Data
(60) Provisional application No. 63/401,395, filed on Aug. 26, 2022.

(51) Int. Cl. A61K 38/04; A61K 9/00; A61K 9/10; A61K 47/26; A61P 27/02

(52) U.S. Cl. CPC A61K 38/04; A61K 9/0019; A61K 9/0048; A61K 9/10; A61K 47/26; A61P 27/02

(57) ABSTRACT

The present disclosure provides pharmaceutical compositions that comprise a microparticle or nanoparticle suspension of a collagen IV-derived peptide or pharmaceutically-acceptable salt thereof, as well as uses of these suspensions for therapy by intraocular delivery. The present disclosure further provides methods for making the microparticle or nanoparticle suspensions.

### PRIORITY

The present application claims the benefit of, and priority to, U.S. provisional application No. 63/401,395 filed Aug. 26, 2022, the contents of which are hereby incorporated by reference in its entirety.

### SEQUENCE LISTING

The instant application contains a sequence listing, which has been submitted in XML format via EFS-Web. The contents of the XML copy named “114293-5018_Sequence_Listing”, which was created on Aug. 25, 2023, and is 37,000 bytes in size, the contents of which are incorporated herein by reference in their entirety.

### BACKGROUND

Diabetic macular edema (DME) and wet age-related macular degeneration (AMD) are the leading causes of blindness among adults in the developed world. There are an estimated 200 million people with AMD in the world, and this may increase to 300 million worldwide by 2040. Progression of AMD leads to blurriness and erosion of central vision, which is necessary for many normal day-to-day activities, such as walking, reading, driving and identifying faces. In dry AMD, light-sensitive cells in the macula become thinner and eventually die, leading to gradual loss of vision and often complete blindness. For wet AMD, blood vessels grow from underneath the macula, and the blood vessels leak blood and fluid into the retina, and which causes the vision to become distorted so that straight lines look wavy. Wet AMD leads to faster vision loss and is the most advanced form of the disease.

Currently, there are several intravitreal anti-VEGF drugs on the market that are typically the first-line treatment option. These drugs have lowered the incidence of AMD-related blindness, and allow many patients to maintain vision. These include anti-VEGF drugs include ranibizumab (LUCENTIS) and aflibercept (EYLEA). During treatment, the ophthalmologist places anesthetic and antiseptic drops on the eye, then administers the anti-VEGF drug by injection into the vitreous (e.g., near the retina at the back of the eye). However, one shortcoming is the recommended frequency of these injections varies from every few weeks to every few months. Patients often require multiple doses over the course of a few months, and repeat treatments are needed for continued benefit. The cost per treatment can be very high, around $2000 per injection in the U.S. Further, potential complications of intravitreal injection include infection (such as that caused by Streptococcus endophthalmitis), retinal detachment, ocular hypertension, cataracts, inflammation, and others. These issues result in low compliance and under treatment which ultimately leads to worsening of disease and visual loss.

Accordingly, potent, safe, and patient-friendly therapies for eye disease, including retinal disease such as wet AMD and diabetic macular edema, are desirable.

### SUMMARY OF THE INVENTION

The present disclosure provides pharmaceutical compositions that comprise a microparticle or nanoparticle suspension of a collagen IV-derived peptide or a pharmaceutically-acceptable salt thereof, as well as uses of these suspensions for therapy by intraocular delivery. The present disclosure further provides methods for making the microparticle or nanoparticle suspensions.

Microparticulate and nanoparticulate suspensions of collagen IV-derived peptides (such as but not limited to the peptide known as AXT107) are described herein for treating ocular diseases. The particles in these suspensions can be within a defined size range and reproducibly made using the methods disclosed herein. In addition, the suspensions can be administered into the suprachoroidal space thereby providing a safe and effective means for delivering collagen-IV-derived peptides for treatment of ocular disease. Further, the microparticulate and nanoparticulate suspensions in various embodiments exhibit a long duration of sustained release in vivo, and therefore can be administered quite infrequently, compared to the standard of care with anti-VEGF drugs such as ranibizumab (LUCENTIS) and aflibercept (EYLEA).

Accordingly, in one aspect, the present disclosure provides a pharmaceutical composition comprising a microparticle or nanoparticle suspension of a collagen IV-derived peptide or pharmaceutically-acceptable salt thereof. In accordance with aspects of the invention, the collagen IV-derived peptide promotes the Tie2 agonist activities of Angiopoietin 2 (Ang2), thereby stabilizing vasculature and providing anti-inflammatory action. The collagen IV-derived peptides are derived from the α5 fibril of type IV collagen. The peptides target and disrupt α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and epidermal growth factor receptor (EGFR). In some embodiments, the collagen IV-derived peptide comprises the amino acid sequence LRRFSTAPFAFIDINDVINF (SEQ ID NO: 3), or derivative thereof.

In accordance with this disclosure, an effective amount of the composition is administered as one or more unit doses that provides a long duration of action, which substantially reduces the frequency of required injections. For example, two or more unit doses are administered by intraocular injection no more frequently than about once every four months or once every six months. That is, at least two consecutive unit doses are spaced in time by at least four months or by at least six months.

In various embodiments, the concentration of the collagen IV-derived peptide in the suspension is from about 1 mg/mL to about 15 mg/mL, or from about 5 mg/mL to about 10 mg/mL. In some embodiments, the concentration of the collagen IV-derived peptide is about 5 mg/mL, or about 7.5 mg/mL, or about 10 mg/mL.

In various embodiments, the composition comprises physiological salts, such as chloride salts of sodium, potassium, calcium, and/or magnesium. In various embodiments, the composition comprises physiological sodium chloride solution, such as about 0.9% sodium chloride (about 154 mM). The suspension may include other excipients and/or carriers including preservatives, physiologically acceptable buffering agents, and biologically acceptable salts so long as the peptide agents are maintained in the suspension form. In some embodiments, excipients are selected to match the tonicity of the vitreous. Exemplary excipients for adjusting tonicity include saccharides, such as monosaccharides and/or disaccharides such as but not limited to sucrose, dextrose, trehalose, and mannitol. In exemplary embodiments, the unit dose comprises about 1% to about 10% by weight of a tonicity adjusting saccharide such as sucrose. For example, the solution may comprise about 5% sucrose.

To maintain the peptide in particulate form, the pH of the composition will have a pH in the range of 6.0 to about 8.0. In exemplary embodiments, the pH is a physiological pH, such as about 7.0 or about 7.4.

In some embodiments, the pharmaceutical composition further comprises hyaluronic acid or a hyaluronic acid derivative, or a pharmaceutically acceptable salt thereof. In some embodiments, the addition of hyaluronic acid (or derivative thereof) results in a slower release of the collagen IV-derived peptide, thereby reducing the needed frequency of administration. In exemplary embodiments, the composition comprises a low molecular weight hyaluronic acid (e.g., sodium hyaluronate).

In some embodiments, the composition further comprises cellulose or a cellulose derivative, or pharmaceutically acceptable salt thereof. In some embodiments, the composition comprises carboxymethylcellulose (e.g., sodium carboxymethylcellulose).

In various embodiments, the suspension is a microparticle suspension. In some embodiments, the microparticle suspension consists of, or consists essentially of, 5 mg/ml to 10 mg/ml API (e.g., AXT107), 5% sucrose, and 0.9% sodium chloride (154 mM).

Alternatively, the suspension is a nanoparticle suspension. For example, the composition may comprise sodium hyaluronate (e.g., sodium salt of a low molecular weight hyaluronic acid) and carboxymethylcellulose (sodium carboxymethylcellulose), which allows for particles in the nanoscale. Nanoparticle suspensions are believed to provide benefits such as more rapid release of the active agent (where desired), as well as benefits in stability of the suspension such as particle settling kinetics.

The pharmaceutical compositions described herein can be provided in unit dose forms suitable for intraocular injection (e.g., suprachoroidal injection). The unit doses comprise about 10 μg to about 1 mg of a collagen IV-derived peptide or salt thereof in a pre-filled syringe. In some embodiments, the composition is a unit dose of from about 100 μg to about 750 μg. Exemplary unit doses include about 100 μg, about 250 μg, about 500 μg, and about 750 μg. The unit dose volume may be in the range of about 1 μL to about 100 μL. In various embodiments, the volume is about 25 μL, about 50 μL, or about 75 μL.

In various embodiments, the composition comprises an additional therapeutic agent or imaging agent, which is optionally conjugated to the peptide, through a bond that is optionally physiologically cleavable or hydrolysable. In these embodiments, the therapeutic agent is trapped by the suspension, and released over time. Exemplary therapeutic agents include proteins, peptides, small molecules, polymers, polynucleotides, oligonucleotides, aptamers, carbohydrates, and lipids. In some embodiments, the collagen IV-derived peptide is designed to be biologically inert, but acts as a carrier for sustained release of the therapeutic agent.

According to certain aspects, the disclosure provides a method (or use) that comprises administering an effective amount of the pharmaceutical composition described herein to a subject in need thereof. In some embodiments, the subject is a human subject, but may also be a non-human mammal in some embodiments. The pharmaceutical composition is generally administered by intraocular administration. In some embodiments, the ocular condition is selected from age-related macular degeneration (AMD), diabetic macular edema (DME), diabetic retinopathy, macular edema (ME), neovascular glaucoma, and retinopathy of prematurity. In some embodiments, the subject has a condition selected from dry or wet AMD. In some embodiments, the subject has dry AMD and geographic atrophy.

In various embodiments, the pharmaceutical composition is administered by suprachoroidal injection. For suprachoroidal delivery, the pharmaceutical composition is delivered to the space between the sclera and the choroid. The composition travels circumferentially and posteriorly in the suprachoroidal space. Devices and methods for suprachoroidal injection are known.

The microparticle or nanoparticle suspension will provide for a long duration of action. In some embodiments, the administration is no more frequent than once every six months, or no more frequent than about once every nine months, or no more frequent than once every year. In exemplary embodiments, the administration frequency is about once every six months. For example, the majority of injections are given about six months apart.

In another aspect, the present disclosure provides a method for treating an ocular condition characterized by inflammation and/or neovascularization, comprising, administering an effective amount of a pharmaceutical composition comprising a collagen IV-derived peptide by suprachoroidal administration. In this aspect, the composition can be a microparticle or nanoparticle suspension as described herein, or other formulations providing for immediate bioavailability or sustained release. In accordance with this disclosure, the collagen IV-derived peptide has good bioavailability in the eye when administered by suprachoroidal injection.

In other aspects, the present disclosure provides methods for making the microparticle or nanoparticle suspension of a collagen IV-derived peptide. The method comprises preparing a solution of the collagen IV-derived peptide, and mixing the solution of the collagen IV-derived peptide with a physiological salt solution (such as a sodium chloride solution), to prepare the microparticle or nanoparticle suspension. In some embodiments, the peptide solution is an aqueous solution that is optionally buffered, for example, within the pH range of about 2.0 to about 5.0, to drive or maintain the peptide in solution form. In some embodiments, the low pH is obtained by the addition of HCl (e.g., 5 mM HCl).

In some embodiments, the collagen IV-derived peptide solution is mixed with a sodium chloride solution to prepare a final solution of 0.9% sodium chloride. The sodium chloride solution will further comprise sodium hydroxide to achieve the resulting pH of the suspension (pH of 6.0 to 8.0 or as already described). For example, the sodium chloride solution can contain 5 mM NaOH when used in equal volumes with the peptide solution. In various embodiments, the solution of the collagen IV-derived peptide and the sodium chloride solution are mixed for several hours to prepare the microparticle or nanoparticle suspension. The solutions can be mixed with stirring, for example at 500 to 2000 rpm (e.g., about 1200 rpm).

Other aspects and embodiments will be apparent from the following detailed description.

### DESCRIPTION OF THE FIGURES

FIG. 1 shows a fluorescent microscopic image of a microparticle suspension created by precipitating AXT107 with sodium chloride. AXT107 microparticles were visualized in a hemocytometer by fluorescence microscopy after addition of DAPI. FIG. 1 shows a 10 mg/mL suspension of AXT107 containing 5% sucrose and 0.9% NaCl.

FIG. 2A, FIG. 2B, and FIG. 2C are graphs and images comparing the settling rate of suspensions made with hyaluronic acid (FIG. 2C) and without hyaluronic acid (FIG. 2B). FIG. 2A compares the settling rates of the two formulations.

FIG. 3 is a fluorescent microscopic image showing microparticle suspension of AXT107 created by precipitating AXT107 with sodium hyaluronate (NaHA) and NaCl. AXT107 microparticles were visualized in a hemocytometer by fluorescence microscopy after addition of DAPI. FIG. 3 shows a 5 mg/mL suspension of AXT107 containing 9 mg/mL HA, 5% sucrose, and 0.9% NaCl.

FIG. 4 are fluorescent microscopic images showing AXT107 microparticles. FIG. 4 shows colocalization of FAM-AXT107, and unlabeled AXT107 in microparticles. DAPI was used to visualize the AXT107 microparticles, and the microparticles were also visualized by monitoring the fluorescence of FAM-AXT107. In this experiment, 5 mg/mL AXT107 (4.5 mg/mL unlabeled AXT107+0.5 mg/mL FAM-AXT107) was dissolved in 5% sucrose solution in the presence of 2.5% DMSO. The AXT107 was precipitated by the addition of 9 mg/mL HA and 0.9% NaCl and microparticles were generated by stirring for 18 hours.

FIG. 5 is a fluorescent microscopic image showing AXT107 microparticles. FIG. 5 shows colocalization of FITC-BSA and unlabeled AXT107 in microparticles. DAPI was used to visualize the AXT107 microparticles, and the microparticles were also visualized by monitoring the fluorescence of FITC-BSA. In this experiment, 10 mg/mL AXT107 and 1 mg/mL FITC-BSA was dissolved in 5% sucrose solution. The AXT107 was precipitated by the addition of 0.9% NaCl and microparticles were generated by stirring for 18 hours.

FIG. 6 is a graph showing inhibition of cMet signaling by AXT107 suspensions. Multiple formulations of AXT107 microparticle and nanoparticle suspensions at a concentration of 100 μM inhibit c-Met phosphorylation in endothelial cells (HUVEC cells) induced by hepatocyte growth factor (HGF) (50 ng/mL). Graphed values indicate the levels of phosphorylated cMet normalized to GAPDH as a loading control in HUVEC lysates presented as a percentage relative to cells stimulated with HGF alone. Except for “untreated,” all samples were stimulated with HGF in addition to the indicated suspension or solution formulation. From left to right the compositions were as follows: soluble AXT107 in 5% sucrose, M15 (AXT107 in 5% sucrose; 9 mg/ml hyaluronic acid, 4 kDa MW, and disrupted into particles by sonication), M16 (AXT107 in 5% sucrose; 9 mg/ml sodium hyaluronate, 4 kDa MW; 0.9% sodium chloride and disrupted into particles by sonication), M20 (AXT107 in 5% sucrose; 9 mg/ml sodium hyaluronate, 4 kDa MW; 0.5% carboxymethyl cellulose; 0.9% sodium chloride and disrupted into particles by sonication), M27 (AXT107 in 5% sucrose; 9 mg/ml sodium hyaluronate, 4 kDa MW; 0.9% sodium chloride and disrupted into particles by stir bar); M103 (AXT107 in 5% sucrose, 0.9% NaCl and disrupted by stirring). M20 was a nano-sized formulation, while all others were in the micro range.

FIG. 7 shows a fluorescent microscopic image of endothelial cells (HUVEC) untreated (top panels) or treated with the AXT107 microparticulate suspension M103 (AXT107 in 5% sucrose precipitated by 0.9% sodium chloride and disrupted into particles by stir bar) (bottom panels). Cells were stained using antibodies against the cell-junction marker VE-cadherin, actin, and nucleus marker DAPI. Scale bar indicates 25 μm.

FIG. 8 is a Western blot image comparing inhibition of cMet phosphorylation by AXT107 solution and M103 suspension. Endothelial cells (HUVEC) were treated with 25 μM AXT107 solution or M103, and inhibition of c-Met phosphorylation induced by hepatocyte growth factor (HGF ((50 ng/ml) was determined. GAPDH is a loading control. Numbers beneath the image indicate levels of phosphorylated cMet normalized to GAPDH as a loading control presented as a percentage relative to cells stimulated with HGF alone (second lane).

### DETAILED DESCRIPTION

The present disclosure provides pharmaceutical compositions that comprise a microparticle or nanoparticle suspension of a collagen IV-derived peptide or pharmaceutically-acceptable salt thereof, as well as uses of these suspensions for therapy by intraocular delivery. The present disclosure further provides methods for making the microparticle or nanoparticle suspensions.

Certain collagen IV-derived peptides, such as the peptide known as AXT107 or gersizangitide, precipitate to form a gel-like substance when administered into vitreous from animals and people. See WO 2020/198481 and US 2019/0225670, which are hereby incorporated by reference in their entireties. The gel is formed when an aqueous solution of AXT107 hits the physiological pH, salt, and the viscosity of the hyaluronic acid of the vitreous. While this gelling features provides the basis for efficiently delivering a depot of therapeutic peptide into the vitreous by simple injection of an aqueous solution, it has been discovered that the properties of the gel formed in the vitreous have certain undesirable properties. For example, the gel can potentially spread into different parts of the vitreous resulting in floaters and fragments, which can break off and move to the anterior chamber and cause an increase in intraocular pressure. Further, the properties of the vitreous can affect the physical properties of the gel, making it difficult to control the pharmacodynamics with intravitreal injection from patient-to-patient. For example, vitreous can be heterogeneous with distinct liquid and more solid regions, the vitreous between patients can also be different, and the effect of diseases such as diabetic macular edema and age-related macular degeneration on the vitreous is incompletely understood. These factors make intravitreal administration of the aqueous peptide solution not therapeutically desirable.

As described herein, microparticulate and nanoparticulate suspensions of collagen IV-derived peptides (such as but not limited to AXT107) are developed to realize the therapeutic potential of these peptides for treating ocular diseases. The particles in these suspensions can be within a defined size range and reproducibly made using the methods disclosed herein. In addition, the suspensions can be administered into the suprachoroidal space, instead of intravitreal administration, thereby providing a safe and effective means for delivering collagen-IV-derived peptides for treatment of ocular disease. Further, the microparticulate and nanoparticulate suspensions in various embodiments exhibit a long duration of sustained release in vivo, and therefore can be administered quite infrequently, compared to the standard of care with anti-VEGF drugs such as ranibizumab (LUCENTIS) and aflibercept (EYLEA).

Accordingly, in one aspect, the present disclosure provides a pharmaceutical composition comprising a microparticle or nanoparticle suspension of a collagen IV-derived peptide or pharmaceutically-acceptable salt thereof.

Exemplary collagen IV-derived peptides comprise the amino acid sequence LRRFSTXPXXXXDINDVXNF (SEQ ID NO: 1), where X is a standard amino acid or a non-genetically-encoded amino acid. Alternatively, the collagen IV-derived peptide comprises the amino acid sequence LRRFSTXPXXXXNINNVXNF (SEQ ID NO: 2), where X is a standard amino acid or a non-genetically-encoded amino acid. In certain embodiments, the peptide comprises or consists of the amino acid sequence LRRFSTAPFAFIDINDVINF (SEQ ID NO: 3) (also known as AXT107 or gersizangitide) or LRRFSTAPFAFININNVINF (SEQ ID NO: 4).

In accordance with aspects of the invention, the collagen IV-derived peptide promotes the Tie2 agonist activities of Angiopoietin 2 (Ang2), thereby stabilizing vasculature and providing anti-inflammatory action. See US 2019/0225670, which is hereby incorporated by reference in its entirety. The collagen IV-derived peptides are derived from the α5 fibril of type IV collagen. The peptides target and disrupt α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and epidermal growth factor receptor (EGFR).

Collagen IV-derived peptides further include those described in U.S. Pat. Nos. 9,056,923 and 9,802,984, which are hereby incorporated by reference in their entireties. For example, peptides in accordance with the following disclosure include peptides comprising the amino acid sequence LRRFSTXPXXXXNINNVXNF (SEQ ID NO: 2), where X at position 7 is M, A, or G; X at position 9 is F, A, Y, or G; X at position 10 is M, A, G, D-Alanine (dA), or norleucine (Nle); X at position 11 is F, A, Y, G, or 4-chlorophenylalanine (4-ClPhe); and X at position 12 and position 18 are independently selected from 2-Aminobutyric acid (Abu), G, S, A, V, T, I, L, or Allylglycine (AllylGly). In various embodiments, the peptide contains about 30 amino acids or less, or about 25 amino acids of less, or about 24 amino acids, or about 23 amino acids, or about 22 amino acids, or about 21 amino acids, or about 20 amino acids. In still other embodiments, one, two, three, four, or five amino acids of SEQ ID NO: 2 are deleted. In some embodiments, the peptide comprises or consists of the amino acid sequence LRRFSTAPFAFININNVINF (SEQ ID NO: 4).

In some embodiments, the peptide comprises the amino acid sequence LRRFSTAPFAFIDINDVINF (SEQ ID NO: 3), or derivative thereof. Derivatives of the peptide of SEQ ID NO: 3 include peptides having from 1 to 5 amino acid substitutions, insertions, or deletions (e.g., 1, 2, 3, 4, or 5 amino acid substitutions, insertions, or deletions collectively) with respect to SEQ ID NO: 3, although in some embodiments the Asp at positions 13 and 16 are maintained. In some embodiments, the sequence DINDV is maintained in the derivative. The peptide may have the amino acid sequence of LRRFSTXPXXXXDINDVXNF, where X at position 7 is M, A, or G; X at position 9 is F, A, Y, or G; X at position 10 is M, A, G, D-Alanine (dA), or norleucine (Nle); X at position 11 is F, A, Y, G, or 4-chlorophenylalanine (4-ClPhe); and X at position 12 and position 18 are independently selected from 2-Aminobutyric acid (Abu), G, S, A, V, T, I, L, or Allylglycine (AllylGly). In various embodiments, the peptide contains about 30 amino acids or less, or about 25 amino acids of less, or about 24 amino acids, or about 23 amino acids, or about 22 amino acids, or about 21 amino acids, or about 20 amino acids. In still other embodiments, one, two, three, four, or five amino acids of SEQ ID NO: 1 or SEQ ID NO: 3 are deleted.

In some embodiments, amino acid substitutions are made at any position of a peptide of SEQ ID NOS: 1 to 4, which can be independently selected from conservative or non-conservative substitutions. In these or other embodiments, the peptide includes from 1 to 10 amino acids added to one or both termini (collectively). The N- and/or C-termini may optionally be occupied by another chemical group (other than amine or carboxy, e.g., amide or thiol).

Conservative substitutions may be made, for instance, on the basis of similarity in polarity, charge, size, solubility, hydrophobicity, hydrophilicity, and/or the amphipathic nature of the amino acid residues involved. The 20 genetically encoded amino acids can be grouped into the following six standard amino acid groups:
(1) hydrophobic: Met, Ala, Val, Leu, Ile;
(2) neutral hydrophilic: Cys, Ser, Thr; Asn, Gln;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (iii) Asn and Gln; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

In various embodiments, the peptide is from about 8 to about 30 amino acids in length, or from about 10 to about 20 amino acids in length, and has at least 4, at least 5, or at least 6 contiguous amino acids of SEQ ID NO: 3 or 4. In some embodiments, the peptide contains at least one, at least two, or at least three d-amino acids. In some embodiments, the peptide contains from one to about five (e.g., 1, 2, or 3) non-genetically encoded amino acids, which are optionally selected from 2-Aminobutyric acid (Abu), norleucine (Nle), 4-chlorophenylalanine (4-ClPhe), and Allylglycine (AllylGly). In some embodiments, the peptide is the retroinverso peptide of a peptide described herein (including the peptide of SEQ ID NO: 3 or SEQ ID NO: 4).

Exemplary peptides in accordance with the disclosure include:

(SEQ ID NO: 5)
LRRFSTMPFMF(Abu)NINNV(Abu)NF,
(SEQ ID NO: 6)
LRRFSTMPAMF(Abu)NINNV(Abu)NF,
(SEQ ID NO: 7)
LRRFSTMPFAF(Abu)NINNV(Abu)NF,
(SEQ ID NO: 8)
LRRFSTMPFMA(Abu)NINNV(Abu)NF,
(SEQ ID NO: 9)
LRRFSTMPF(Nle)F(Abu)NINNV(Abu)NF,
(SEQ ID NO: 10)
LRRFSTMPFM(4-ClPhe)(Abu)NINNV(Abu)NF,
(SEQ ID NO: 11)
LRRFSTMPFMFSNINNVSNF,
(SEQ ID NO: 12)
LRRFSTMPFMFANINNVANF,
(SEQ ID NO: 13)
LRRFSTMPFMFININNVINF,
(SEQ ID NO: 14)
LRRFSTMPFMFTNINNVTNF,
(SEQ ID NO: 15)
LRRFSTMPFMF(AllyGly)NINNV(AllyGly)NF,
(SEQ ID NO: 16)
LRRFSTMPFMFVNINNVVNF,
(SEQ ID NO: 17)
LRRFSTMPFdAFININNVINF,
(SEQ ID NO: 18)
LRRFSTMPFAFININNVINF,
(SEQ ID NO: 19)
LRRFSTAPFAFININNVINF,
(SEQ ID NO: 20)
LRRFSTAPFdAFIDINDVINF,
(SEQ ID NO: 21)
LRRFSTAPFAFIDINDVINW,
(SEQ ID NO: 22)
dLRRdLRRFSTAPFAFIDINDVINF,
(SEQ ID NO: 23)
LRRFSTAPFAFIDINDVINdF,
(SEQ ID NO: 24)
dLRRFSTAPFAFIDINDVINdF.
(SEQ ID NO: 25)
F(Abu)NINNV(Abu)N,
(SEQ ID NO: 26)
FTNINNVTN,
(SEQ ID NO: 27)
FININNVINF,
(SEQ ID NO: 28)
FSNINNVSNF,
(SEQ ID NO: 29)
FANINNVANF,
(SEQ ID NO: 30)
F(AllyGly)NINNV(AllyGly)NF,
(SEQ ID NO: 31)
FVNINNVVNF,
(SEQ ID NO: 32)
FIDINDVINF,
(SEQ ID NO: 33)
FIDINDVINW,
(SEQ ID NO: 34)
FTDINDVTN,
(SEQ ID NO: 35)
A(Abu)NINNV(Abu)NF,
or
(SEQ ID NO: 36)
(4-ClPhe)(Abu)NINNV(Abu)NF.

In various embodiments, the peptide forms a gel in mock vitreous (e.g., physiological pH and salt in the presence of hyaluronic acid).

The peptide in some embodiments is provided as a pharmaceutically acceptable salt. Pharmaceutically acceptable peptide salts are generally well known to those of ordinary skill in the art, and may include, by way of example, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, and teoclate. Other pharmaceutically acceptable salts may be found in, for example, Remington: The Science and Practice of Pharmacy (20th ed.) Lippincott, Williams & Wilkins (2000). In some embodiments, the peptide is a hydrochloride salt.

The peptides can be chemically synthesized and purified using well-known techniques, such as solid-phase synthesis. See U.S. Pat. No. 9,051,349, which is hereby incorporated by reference in its entirety.

In accordance with this aspect, the pharmaceutical composition comprises a microparticle or nanoparticle suspension of the collagen IV-derived peptide. The term “microparticle or nanoparticle suspension” means that the collagen IV-derived peptide is not substantially or predominately in solution state, and has been precipitated to form particulates (e.g., by addition of physiological salt and/or raising of the pH to physiological pH, and/or increasing viscosity such as with hyaluronic acid), together with certain excipients or co-agents as described herein. The microparticle and nanoparticle suspensions are physically distinct from conjugation to or encapsulation of the peptide by polymer beads or particles (for example, as described in US 2020/0179285, which is hereby incorporated by reference). That is, the suspensions do not include polymers such as PLGA or PLGA-PEG or similar polymers (e.g., PLA, PLA-PEG, poly-β-amino esters, or others). The suspensions do not comprise liposomes or lipid nanoparticles, or similar encapsulation agents.

In accordance with this disclosure, an effective amount of the composition is administered as one or more unit doses that provides a surprisingly long duration of action, which substantially reduces the frequency of required injections. For example, two or more unit doses are administered by intraocular injection no more frequently than about once every four months. That is, at least two consecutive unit doses are spaced in time by at least four months. Further, in accordance with this disclosure, the peptide is delivered without the use of advanced formulation technologies (e.g., polymeric particle encapsulation), such as nanoparticle or microparticle encapsulation, or liposome encapsulation. Other half-life extension or stabilization technologies compatible with peptides, such as pegylation, acylation, or fusion to carrier proteins (e.g., Fc domain or albumin), are unnecessary and need not be employed.

In various embodiments, the concentration of the collagen IV-derived peptide in the suspension is from about 1 mg/mL to about 15 mg/mL, or from about 2 mg/mL to about 12 mg/mL, or from about 4 mg/mL to about 12 mg/mL, or from about 2 mg/mL to about 10 mg/mL, or from about 2 mg/mL to about 10 mg/mL, or from about 5 mg/mL to about 10 mg/mL. In various embodiments, the concentration of the collagen IV-derived peptide is about 5 mg/mL, or about 7.5 mg/mL, or about 10 mg/mL.

In various embodiments, the composition comprises physiological salts, such as chloride salts of sodium, potassium, calcium, and/or magnesium. In various embodiments, the composition comprises physiological sodium chloride solution, such as about 0.9% sodium chloride (about 154 mM). The suspension may include other excipients and/or carriers including preservatives, physiologically acceptable buffering agents, and biologically acceptable salts so long as the peptide agents are maintained in the suspension form. In some embodiments, excipients are selected to match the tonicity of the vitreous (e.g., osmolality of from about 250 to about 350 mOsm). Exemplary excipients for adjusting tonicity include saccharides, such as monosaccharides and/or disaccharides such as but not limited to sucrose, dextrose, trehalose, and mannitol. In exemplary embodiments, the unit dose comprises about 1% to about 10% by weight of a tonicity adjusting saccharide (such as sucrose or dextrose), such as about 2% to about 8% by weight, or about 5% by weight of the tonicity adjusting saccharide (such as sucrose and/or dextrose). For example, the solution may comprise about 5% sucrose.

The composition may optionally comprise additional excipients, including cryoprotectants. An exemplary cryoprotectant is dimethyl sulfoxide (DMSO). Other excipients that can be included in certain embodiments include (without limitation) ethylene glycol, glycerol, propylene glycol, and polyethylene glycol.

To maintain the peptide in particulate form, the pH of the composition will have a pH in the range of 6.0 to about 8.0, or in the range of 6.5 to about 8.0, or in the range of about 6.8 to about 7.8. In exemplary embodiments, the pH is a physiological pH, such as about 7.0 or about 7.4.

In various embodiments, the suspension is optionally pH buffered. Exemplary buffering agents include bicarbonate (sodium bicarbonate) or phosphate buffer (e.g., sodium phosphate buffer).

In some embodiments, the pharmaceutical composition further comprises hyaluronic acid or a hyaluronic acid derivative, or a pharmaceutically acceptable salt thereof. Hyaluronic acid derivatives can include any chemical conjugation to available functional groups, including inert chemical conjugations that alter the physical properties of the hyaluronic acid or conjugation of active agents (including active agents described herein). In some embodiments, the hyaluronic acid is crosslinked. In some embodiments, the addition of hyaluronic acid (or derivative thereof) results in a slower release of the collagen IV-derived peptide, thereby reducing the needed frequency of administration. In exemplary embodiments, the composition comprises a low molecular weight hyaluronic acid (e.g., sodium hyaluronate) at a concentration of at least 0.5 mg/mL, or at least about 1 mg/mL, or at least about 4 mg/mL, or at least about 8 mg/mL. In some embodiment, the composition comprises hyaluronic acid (e.g., sodium hyaluronate) at a concentration of from about 1 mg/mL to about 15 mg/mL, or from about 2 mg/mL to about 10 mg/mL, such as about 4.5 mg/mL or about 9 mg/mL. In some embodiments, the low molecular weight hyaluronic acid has a molecular weight of less than about 10 kDa, or less than about 8 kDa, such as about 4 kDa.

In some embodiments, the composition further comprises cellulose or a cellulose derivative, or pharmaceutically acceptable salt thereof. Exemplary cellulose derivatives include methylcellulose, hydroxypropyl cellulose, and carboxymethylcellulose. In some embodiments, the composition comprises from about 0.01% to about 5.0% or from about 0.1% to about 2%, or from about 0.1% to about 1% carboxymethylcellulose (e.g., sodium carboxymethylcellulose). Exemplary formulations have from 0.2% to about 0.9% sodium carboxymethylcellulose.

In various embodiments, the suspension is a microparticle suspension where the majority of particles have a size in the range of about 1 to about 200 microns (i.e., measured as a diameter or the longest dimension of the particle), or in the range of about 1 micron to about 100 microns, or about 1 micron to about 50 microns. In some embodiments, the majority of particles in the microparticle suspension have a particle size of at least about 1 micron, or at least about 10 microns, or at least about 20 microns. In various embodiments, the majority particles are in the range of about 10 to about 100 microns. In some embodiments, the microparticle suspension comprises low molecular weight hyaluronic acid (e.g., sodium hyaluronate) or carboxymethylcellulose (e.g., sodium carboxymethylcellulose) as described (but not both). In still other embodiments, the microparticle suspension does not contain hyaluronic acid or a derivative thereof or a cellulose or derivative thereof. In some embodiments, the composition consists essentially of, or consists of, the collagen IV-derived peptide (or salt thereof), physiological salt(s) such as sodium chloride, and monosaccharide or disaccharide tonicity agent (such as sucrose) in water (according to concentrations already described) and optionally hyaluronic acid, carboxymethylcellulose, and/or a cryoprotectant such as DMSO. In some embodiments, the microparticle suspension consists of, or consists essentially of, 5 mg/mL to 10 mg/ml API (e.g., AXT107), 5% sucrose, and 0.9% sodium chloride (154 mM). In this context, the term “consists essentially of” means that additional excipients can be added as long as they do not significantly impact properties of the suspension such as physiological compatibility, particle size, release kinetics of the collagen type IV-derived peptide, and particle settling kinetics.

Alternatively, the suspension is a nanoparticle suspension. For example, the composition may comprise sodium hyaluronate (e.g., sodium salt of a low molecular weight hyaluronic acid) and carboxymethylcellulose (sodium carboxymethylcellulose), which allows for particles in the nanoscale (i.e., majority of particles in the suspension are less than one micron in diameter or the longest dimension is less than one micron). Nanoparticle suspensions are believed to provide benefits such as more rapid release of the active agent (where desired), as well as benefits in stability of the suspension such as particle settling kinetics.

The pharmaceutical compositions described herein can be provided in unit dose forms suitable for intraocular injection (e.g., suprachoroidal injection or intravitreal injection). The unit doses comprise about 10 μg to about 1 mg of a collagen IV-derived peptide or salt thereof (as disclosed herein) in a pre-filled syringe. For example, the unit dose may be about 750 μg or less of the peptide or salt thereof, or about 500 μg or less of the peptide or salt thereof, or about 250 μg or less of the peptide or salt thereof, or about 100 μg or less of the peptide or salt thereof. In some embodiments, the composition is a unit dose of from about 100 μg to about 750 μg, or about 250 μg to about 750 μg, or about 400 μg to about 750 μg, or about 500 μg to about 750 μg of the peptide or salt thereof. Exemplary unit doses include about 100 μg, about 250 μg, about 500 μg, and about 750 μg.

The unit dose volume (in accordance with the compositions and methods described herein) may be in the range of about 1 μL to about 100 μL, or from about 10 μL to about 100 μL, or from about 10 μL to about 75 μL, or from about 10 μL to about 50 μL. In some embodiments, the pharmaceutical composition comprises a unit volume of from about 25 μL to about 100 μL, or a unit volume of from about 50 μL to about 100 μL, or a unit volume of from about 25 μL to about 75 μL. In some embodiments, the unit volume is less than about 100 μL, or less than about 50 μL, or less than about 25 μL. In various embodiments, the volume is about 25 μL, about 50 μL, or about 75 μL.

In various embodiments, the composition comprises an additional therapeutic agent or imaging agent (or visible or detectable label), which is optionally conjugated to the peptide, through a bond that is optionally physiologically cleavable or hydrolysable. In these embodiments, the therapeutic agent is trapped by the suspension, and released over time. Exemplary therapeutic agents include proteins, peptides, small molecules, polymers, polynucleotides, oligonucleotides, aptamers, carbohydrates, and lipids. In some embodiments, the collagen IV-derived peptide is designed to be biologically inert (e.g., by amino acid substitution, deletion, and/or insertion), but acts as a carrier for sustained release of the therapeutic agent, which can be optionally conjugated to the peptide, through a bond that is optionally physiologically cleavable or hydrolysable.

In some embodiments, the protein is an antibody, enzyme, cytokine, or soluble receptor ligand. In some embodiments, the antibody is selected from a monoclonal antibody, a polyclonal antibody, a recombinant antibody, a nanobody, a humanized antibody, a chimeric antibody, a multi-specific antibody, or an antibody fragment thereof. In some embodiments, the antibody fragment is a Fab fragment, a Fab′ fragment, a F(ab′)2 fragment, a Fv fragment, a diabody, or a single chain antibody molecule (e.g., scFv).

In some embodiments, the therapeutic agent is an antibody having therapeutic benefit for ocular conditions, such as wet or dry AMD or geographic atrophy (GA), and which can be optionally selected from GSK933776, THR-317, RO6867461, REGN910, DS7080a, TK001, Brolucizumab, REGN2176-3, and iSONEP.

In some embodiments, the therapeutic agent targets a protein of the complement pathway. The complement pathway is recognized to have three activation pathways, initiated through distinct ligand-receptor interactions. The three pathways include: (1) the classical pathway, (2) the lectin pathway, and (3) the alternative pathway. Initiation of the classical pathway occurs when C1 (C1q in complex with the serine proteases C1r and C1s) interacts with the Fc region of IgG or IgM antibodies attached to antigenic surfaces. In the lectin pathway, mannose-binding lectin (MBL) and ficolins assemble with MBL-associated serine proteases (MASPs). The alternative pathway is induced by C3 hydrolysis, either spontaneously at low rate or enhanced by interaction of C3 with a pathogen's cell surfaces. All three pathways lead to the formation of C3 and C5 convertases, which together amplify the complement response. The outcome of complement activation is the following: (1) opsonization of the target surface by C3b, (2) a boost in inflammation through the generation of anaphylatoxins C3a and C5a and subsequent recruitment of effector cells and (3) formation of the terminal membrane attack complex (MAC), which is responsible for target cell lysis. In addition to these processes, several complement regulatory proteins are able to inhibit complement by inactivation of C3b and C3 convertases, or by preventing successful formation of the MAC.

An imbalance in complement, e.g., excessive complement activation, can have an important pathological significance in an ocular condition (e.g., dry or wet age-related macular degeneration (AMD), geographic atrophy). Accordingly, in some embodiments, the therapeutic agent targets a protein of the complement pathway, optionally wherein the complement pathway target is complement factor 1 (C1), C1s, C1q, C1r, complement factor 2 (C2), complement factor 3 (C3), C3a, C3b, C3bBb, complement factor 4 (C4), C4b2b complement factor 5 (C5), C5a, C5b, C5b-9, factor properdin, complement factor B, complement factor D, complement factor H, or complement factor I.

In some embodiments, the therapeutic agent is an antibody or antibody fragment (or a mimetic thereof) that targets complement factor 3 (C3) or complement factor 5 (C5). In other embodiments, the therapeutic agent is complement factor H, or complement factor I, or a fragment or mimetic thereof. In some embodiments, the therapeutic agent is selected from Pegcetacoplan/APL-2, Cp40-KKK/AMY-106, and CB2782. In some embodiments, the antibody or antibody fragment targets C5. In some embodiments, the antibody is selected from Pexelizumab, Avacincaptad Pegol, Eculizumab, and Tesidolumab/LFG316. In some embodiments, the antibody or antibody fragment targets C5a. In some embodiments, the antibody is TNX-558 or Neutrazumab.

In some embodiments, the therapeutic agent targets complement factor B. In some embodiments, the therapeutic agent is TA106 or Ionis-FB-L_{RX}.

In some embodiments, the therapeutic agent targets complement factor D. In some embodiments, the therapeutic agent is an antibody, such as TNX-234 or Lampalizumab.

In some embodiments, the therapeutic agent targets properdin. In some embodiments, the therapeutic agent is an antibody, such as CLG561.

In various embodiments, the therapeutic agent or the collagen IV-derived peptide is coupled to a labeling group, such as an optical label, or an enzymatic group, which can allow for periodic visualization of the suspension.

In some embodiments, the therapeutic agent is a polynucleotide such as RNA or DNA. In some embodiments, the RNA is a messenger RNA (mRNA), short interfering RNA (siRNA), short hairpin or small hairpin RNA (shRNA), or microRNA (miRNA). The RNA may be an RNA interference molecule, which interferes with or inhibits expression of a target gene or genomic sequence by RNA interference (RNAi). In some embodiments, the polynucleotide is conjugated to the collagen IV-derived peptide.

In various embodiments, the small molecule is a chemotherapeutic agent, or a kinase inhibitor. In these embodiments, the therapeutic agent can have therapeutic benefit for proliferative conditions of the eye, such as ocular rhabdomyosarcoma, choroidal or uveal melanoma, or retinoblastoma. An exemplary kinase inhibitor is axitinib.

In some embodiments, the chemotherapeutic agent is an antibiotic. In some embodiments, the antibiotic is selected from amoxicillin, penicillin, doxycycline, clarithromycin, benzylpenicillin, azithromycin, daptomycin, linezolid, levofloxacin, moxifloxacin, gatifloxcin, gentamicin, macrolides, cephalosporins, azithromycin, amoxillin-potassium ciprofloxacin, cefuroxime, clavulanate, erythromycin, sulfamethoxazole-trimethoprim, doxycycline monohydrate, cefepime, ampicillin, cefpodoxime, ceftriaxone, cefazolin, erythromycin ethylsuccinate, meropenem, piperacillin-tazobactam, amikacin, erythromycin stearate, cefepime in dextrose, doxycycline hyclate, ampicillin-sulbactam, ceftazidime, gemifloxacin, gentamicin sulfate, erythromycin lactobionate, imipenem-cilastatin, cefoxitin, cefditoren pivoxil, ertapenem, doxycycline-benzoyl peroxide, ampicillin-sulbactam, meropenem, cefuroxime, and cefotetan.

In some embodiments, the chemotherapeutic agent is selected from cyclophosphamide, busulfan, improsulfan and piposulfan, benzodopa, carboquone, meturedopa, and uredopa, ethylenimines and methylamelamines including altretamine, triethylenemelamine, trietylenephosphoramide, triethylenethiophosphaoramide and trimethylolomelamime, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, methotrexate and 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytosine arabinoside, dideoxyuridine, doxifluridine, enocitabine, floxuridine, 5-FU, calusterone, dromostanolone propionate, epitiostanol, mepitiostane, testolactone, aminoglutethimide, mitotane, trilostane, folinic acid, aceglatone, aldophosphamide glycoside, aminolevulinic acid, amsacrine, bestrabucil, bisantrene, edatraxate, defofamine, demecolcine, diaziquone, elformithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, PSK, razoxane, sizofuran, spirogermanium, tenuazonic acid, triaziquone, 2,2′,2″-trichlorotriethylamine, urethan, vindesine, dacarbazine, mannomustine, mitobronitol, mitolactol, pipobroman, gacytosine, arabinoside (Ara-C), taxoids, paclitaxel and docetaxel, chlorambucil, gemcitabine, 6-thioguanine, mercaptopurine, cisplatin and carboplatin, vinblastine, platinum, etoposide, ifosfamide, mitomycin C, mitoxantrone, vincristine, vinorelbine, navelbine, novantrone, teniposide, daunomycin, aminopterin, xeloda, ibandronate, CPT11, topoisomerase inhibitor RFS 2000, difluoromethylornithine, retinoic acid, esperamicins, and capecitabine.

In some embodiments, the therapeutic agent is a corticosteroid, such as but not limited to triamcinolone acetonide and corticosteroids known in the art.

In various embodiments, the protein is a cytokine, and the cytokine is an anti-inflammatory cytokine that increases the levels of T regulatory cells (Tregs) (e.g., transforming growth factor beta (TGFβ) and interleukin 10 (IL-10)). In other embodiments, the therapeutic agent is a polypeptide (e.g., antibody or soluble receptor) that inhibits a pro-inflammatory cytokine, such as IL-1β, IL-2 or INF-γ.

In various embodiments, the therapeutic agent is covalently conjugated to the collagen IV-derived peptide. In some embodiments, the collagen IV-derived peptide has one or more substitutions that abrogates the anti-vascular permeability, anti-angiogenic, anti-tumor, or anti-inflammatory properties of the peptide. In some embodiments, the conjugation is through a physiologically cleavable bond, such as a hydrolysable bond or enzymatically cleavable bond. The cleavable bond, in some embodiments, is an ester, amide, or thioester. In some embodiments, the enzymatically cleavable bond is a linkage that is subject to degradation by one or more enzymes. In some embodiments, the hydrolysable cleavable bond is a chemical bond (e.g., a covalent bond) that is substantially stable in water, such that it does not undergo hydrolysis under storage conditions over an extended period of time.

In various embodiments, the collagen IV-derived peptide or salt thereof has no biological activity (e.g., the peptide does not restore Tie2 activation). In some embodiments, the only activity of the collagen IV-derived peptide or salt thereof is to form the suspension. In some embodiments, only the therapeutic agent has biological activity. In some embodiments, the peptide is conjugated to the therapeutic agent.

In other aspects, the invention provides a method for treating an ocular condition characterized by inflammation and/or neovascularization, and use of the formulations described herein for therapy. In various embodiments, the collagen IV-derived peptide promotes the Tie2 agonist activities of Angiopoietin 2 (Ang2), thereby stabilizing vasculature. The peptides target and disrupt α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and epidermal growth factor receptor (EGFR). Accordingly, the peptides disclosed herein provide a potent alternative to VEGF blockade or inhibitor therapy, or potent combination therapy.

According to this aspect, the method comprises administering an effective amount of the pharmaceutical composition described herein to a subject in need thereof. In some embodiments, the subject is a human subject, but may also be a non-human mammal in some embodiments (e.g., horse, dog, cat, pig, etc.). The pharmaceutical composition is generally administered by intraocular administration. In some embodiments, the ocular condition is selected from age-related macular degeneration (AMD), diabetic macular edema (DME), diabetic retinopathy, macular edema (ME), neovascular glaucoma, and retinopathy of prematurity. In some embodiments, the subject has a condition selected from dry or wet AMD. In some embodiments, the subject has dry AMD and geographic atrophy.

In still other embodiments, the subject has uveitis, an ocular condition associated with an autoimmune condition (e.g., autoimmune uveitis), or a proliferative condition such as retinoblastoma, ocular rhabdomyosarcoma, choroidal melanoma, or uveal melanoma.

In various embodiments, the pharmaceutical composition is administered by suprachoroidal injection. For suprachoroidal delivery, the pharmaceutical composition is delivered to the space between the sclera and the choroid. The composition travels circumferentially and posteriorly in the suprachoroidal space. Devices and methods for suprachoroidal injection are described in U.S. Pat. Nos. 8,197,435; 9,539,139; and 9,937,075, each of which is hereby incorporated by reference in its entirety.

For suprachoroidal delivery, the drug is injected by inserting a microneedle into the sclera and infusing a drug formulation through the inserted microneedle and into the suprachoroidal space of the eye. The microneedle is able to precisely deliver the drug into the suprachoroidal space for subsequent local availability to nearby tissues in need of treatment. In various embodiments, the microneedle provides precise control of the depth of insertion into the ocular tissue, so that the microneedle tip can be placed into the suprachoroidal space or in the sclera but near enough to the suprachoroidal space for the infused drug formulation to flow into the suprachoroidal space. Advantageously, this may be accomplished without contacting underlying tissues, such as choroid and retina tissues. It is believed that upon entering the suprachoroidal space the fluid drug formulation flows circumferentially from the insertion site toward the retinochoroidal tissue, macula, and optic nerve in the posterior segment of the eye as well as anteriorly toward the uvea and ciliary body.

As used herein, the term “suprachoroidal space” refers to the potential space in the region of the eye disposed between the sclera and choroid. This region primarily is composed of closely packed layers from each of the two adjacent tissues; however, a space can develop in this region as a result of fluid or other material in the suprachoroidal space and the adjacent tissues. Those skilled in the art will appreciate that the suprachoroidal space frequently is expanded by fluid buildup because of some disease state in the eye or as a result of some trauma or surgical intervention.

For suprachoroidal delivery the depth of insertion of the microneedle into the ocular tissue is precisely controlled. For example, the insertion depth can be limited by the selected length or effective length of the microneedle. The “effective length” is that portion available for tissue insertion, i.e., the length that extends from the base and would be inserted if there were zero tissue deformation. That is, the microneedle may have a length approximately equal to the desired penetration depth. For example, the tip of the microneedle can be inserted through the sclera into the suprachoroidal space without penetrating through the choroid. In some embodiments, the microneedles are designed to have a length longer than the desired penetration depth, but the microneedles are controllably inserted only part way into the tissue. Partial insertion may be controlled by the mechanical properties of the tissue.

Alternatively, the pharmaceutical composition is administered by intravitreal injection. Generally, an intravitreal injection is an injection into the eye, and in particular the vitreous which is a jelly-like fluid that fills the eye. During the procedure, for example as is used conventionally for administering anti-VEGF drugs, the health care provider injects medicine into the vitreous, near the retina at the back of the eye.

The microparticle or nanoparticle suspension will provide for a long duration of action. For example, the pharmaceutical composition is generally administered no more frequent than once every other month. In various embodiments, the administration is no more frequent than once every three months (or at least two doses are separated by at least three months), or no more frequent than once every four months (or at least two doses are separated by at least four months). In some embodiments, the administration is no more frequent than once every six months (or at least two doses are separated by at least six months), or no more frequent than about once every nine months (or at least two doses are separate by at least nine months), or no more frequent than once every year (or at least two doses are separated by at least about one year). In exemplary embodiments, the administration frequency is about once every six months. For example, the majority of injections are given about six months apart.

In another aspect, the present disclosure provides a method for treating an ocular condition characterized by inflammation and/or neovascularization (as already described), comprising, administering an effective amount of a pharmaceutical composition comprising a collagen IV-derived peptide by suprachoroidal administration. In this aspect, the composition can be a microparticle or nanoparticle suspension as described herein, or other formulations providing for immediate bioavailability or sustained release. See, for example, US 2019/0225670 and WO 2020/198481, which are hereby incorporated by reference in their entireties. In accordance with this disclosure, the collagen IV-derived peptide has good bioavailability in the eye when administered by suprachoroidal injection. Frequency of administration can be as already described.

In some embodiments of the methods described herein, the patient has macular edema. Macular edema occurs when fluid and protein deposits collect on or under the macula of the eye (a yellow central area of the retina) and causes it to thicken and swell. The causes of macular edema include chronic or uncontrolled diabetes type 2 (e.g., diabetic retinopathy), in which peripheral blood vessels including those of the retina leak fluid into the retina. Other causes and/or associated disorders include age-related macular degeneration (AMD), chronic uveitis, atherosclerosis, high blood pressure and glaucoma. In some embodiments, the patient has or is at risk of retinal vein occlusion, which can lead to severe damage to the retina and blindness, due to ischemia and edema.

In some embodiments (with regard to methods of treatment or uses described herein) the patient may have AMD, which is optionally wet AMD or in some embodiments dry AMD. In some embodiments, the subject has geographic atrophy (“GA”). Two stages of AMD include nonexudative (dry) and exudative (wet) AMD. Dry AMD is characterized by the presence of GA. GA are lesions with deterioration of the photoreceptors, retinal pigment epithelium (RPE), and choriocapillaris. GA lesions are generally found within the macula (central retina), and may cause scotomas within the central visual field that progressively enlarge. Wet AMD is characterized by an abnormal choroidal neovascularization (CNV) beneath the macula. The CNV causes central vision loss from bleeding, edema, and scarring of retinal tissue. Wet AMD is typically treated with intravitreal injections of medicines (e.g. ranibizumab, aflibercept, bevacizumab). Further, complement plays a key role in AMD pathophysiology. In AMD, the complement system becomes dysregulated and thus, AMD is often considered an immune-mediated disease.

In some embodiments, the composition comprises a complement pathway inhibitor to treat or prevent dry or wet AMD, or GA, and such complement pathway inhibitors are described herein. Examples of complement pathway inhibitors include antibodies against C3 or C5, or complement factor H or complement factor I or fragments thereof.

In various embodiments, the patient will receive a plurality of doses, and in some embodiments, therapy can be continuous at the recommended frequency of injections for disease control or management. In some embodiments, therapy need not be continuous, where symptoms or disease has been substantially alleviated. In various embodiments, the patient receives at least two injections, or at least four injections, or at least six injections, or at least eight injections, or at least ten injections. In some embodiments, injections are provided in a regimen of from four to ten injections.

In various embodiments, the peptide formulation described herein can be delivered for conditions (including macular edema, wet AMD) that are refractory or only partially-responsive to vascular endothelial growth factor (VEGF) blockade or inhibitor therapy. Pharmaceutical agents that block VEGF include aflibercept, bevacizumab, ranibizumab, and ramucirumab, and similar agents, which are administered to slow or block angiogenesis. Other agents that target VEGF-mediated biological activity include kinase inhibitors such as axitinib, pazopanib, sorafenib, sunitinib, ponatinib, lenvatinib, vandetanib, regorafenib, and cabozantinib.

Aflibercept is a biopharmaceutical drug for the treatment of wet macular degeneration (EYLEA). Aflibercept is an inhibitor of VEGF, and is a recombinant fusion protein consisting of vascular endothelial growth factor (VEGF)-binding portions from the extracellular domains of human VEGF receptors 1 and 2, that are fused to the Fc portion of the human IgG1 immunoglobulin. Aflibercept binds to VEGFs and acts like a “VEGF trap”, inhibiting the activity of the vascular endothelial growth factor subtypes VEGF-A and VEGF-B, as well as to placental growth factor (PGF).

Bevacizumab (AVASTIN) is an angiogenesis inhibitor, a drug that slows the growth of new blood vessels. Bevacizumab is a recombinant humanized monoclonal antibody that blocks angiogenesis by inhibiting VEGF-A. Bevacizumab is administered for treating certain metastatic cancers, including colon cancer, lung cancers (e.g., NSCLC), renal cancers, ovarian cancers, breast cancer, and glioblastoma. Bevacizumab can also be used for treatment of eye diseases, including AMD and diabetic retinopathy.

Ranibizumab (LUCENTIS) is a monoclonal antibody fragment (Fab), and is administered for treatment of wet AMD. The drug is injected intravitreally (into the vitreous humour of the eye) about once a month. Ranibizumab is a monoclonal antibody that inhibits angiogenesis by inhibiting VEGF A, similar to Bevacizumab.

The collagen IV-derived peptide composition may be administered after unsuccessful VEGF blockade therapy, that is, where reductions in angiogenesis, lymphangiogenesis, and/or edema were not observed. In some embodiments, the peptide is administered as an alternative to VEGF blockade therapy. In still further embodiments, the peptide is administered in combination with VEGF blockade therapy, either simultaneously with, before, or after a VEGF blockade regimen. By activating Tie2 signaling, the peptide provide therapeutic benefits that may not be observed with VEGF blockage therapy, or VEGF blockade therapy alone.

Therefore, in some embodiments, the formulation described herein is administered after unsuccessful VEGF blockade or inhibitor therapy. In some embodiments, the patient has a condition that is refractory or only partially-responsive to VEGF blockade or inhibitor therapy.

In other aspects, the present disclosure provides methods for making the microparticle or nanoparticle suspension of a collagen IV-derived peptide. The method comprises preparing a solution of the collagen IV-derived peptide, and mixing the solution of the collagen IV-derived peptide with a physiological salt solution (such as a sodium chloride solution), to prepare the microparticle or nanoparticle suspension. In various embodiments, the collagen IV-derived peptide is as described herein, and in some embodiments comprises or consists of the amino acid sequence LRRFSTXPXXXXDINDVXNF (SEQ ID NO: 1) or the amino acid sequence LRRFSTXPXXXXNINNVXNF (SEQ ID NO: 2), where X is a standard amino acid or a non-genetically-encoded amino acid (or other collagen IV-derived peptide described herein). Exemplary collagen IV-derived peptides comprise or consist of the amino acid sequence LRRFSTAPFAFIDINDVINF (SEQ ID NO: 3) or the amino acid sequence LRRFSTAPFAFININNVINF (SEQ ID NO: 4).

In various embodiments, the concentration of the collagen IV-derived peptide in the suspension is from about 1 mg/mL to about 15 mg/mL, or from about 5 mg/mL to about 10 mg/mL. If prepared using equal volumes of the collagen IV-derived peptide solution and the salt solution, the collagen IV-derived peptide solution will have a 2× concentration of the peptide, as compared to the suspension. Of course, in other embodiments, the volume of the salt solution is higher or lower than the collagen IV-derived peptide solution, such as about 0.2× to about 5×, or from about 0.2× to about 3×, or about 0.2× to about 2×. In some embodiments, the solutions are about equal volume (i.e., about 1:1). In some embodiments, the concentration of the peptide in the solution of the collagen IV-derived peptide is from about 2 mg/mL to about 30 mg/mL, or about 2 mg/mL to about 24 mg/mL, or from about 4 mg/mL to about 20 mg/mL, or from about 5 mg/mL to about 20 mg/mL, or from about 10 mg/mL to about 20 mg/mL. Using equal volumes of the two solutions, the collagen IV-derived peptide may be present at a concentration of 10 mg/mL or 20 mg/mL in the collagen IV-derived peptide solution in exemplary embodiments.

The collagen IV-derived peptide solution may contain additional excipients as already described, such as an effective amount of a tonicity agent such as a mono or disaccharide, examples of which include sucrose, trehalose, mannitol, and dextrose. In certain embodiments, such as those using equal volumes of the two solutions, the saccharide (such as sucrose) may be present in the collagen IV-derived peptide solution at about 2% to about 20%, such as from about 4% to about 16% (by weight). For example, the collagen IV-derived peptide solution may comprise about 10% of the stabilizing saccharide (e.g., sucrose).

In some embodiments, the peptide solution is an aqueous solution that is optionally buffered, for example, within the pH range of about 2.0 to about 5.0, to drive or maintain the peptide in solution form. In some embodiments, the low pH is obtained by the addition of HCl (e.g., 5 mM HCl). The peptide solution may further comprise a salt of an organic acid, which is optionally acetate, lactate, malate, succinate, and/or fumarate. The aqueous solution may include other excipients and/or carriers including preservatives and biologically acceptable salts as long as the agents are maintained in solution.

The collagen IV-derived peptide solution (or the physiological salt solution) may optionally comprise additional excipients, including cryoprotectants. An exemplary cryoprotectant is dimethyl sulfoxide (DMSO), which may be present in the range of about 0.5% to about 10% (by vol.), for example. An exemplary concentration of DSMO is 5% (by vol.), providing a concentration in the resulting suspension of about 2.5% (using equal volume of sodium chloride solution). Other cryoprotectants or excipients that can be included in certain embodiments include (without limitation) ethylene glycol, glycerol, propylene glycol, and polyethylene glycol. Excipients (including cryoprotectants and buffering agents described herein) can alternatively be added after mixing of the two solutions (i.e., added to the suspension) to maintain the properties of the final formulation.

The collagen IV-derived peptide solution will have an acidic pH with low salt content to get the peptide in solution. For example, the collagen IV-derived peptide solution will have a pH in various embodiments that is less than 5.0, or in some embodiments about 4.0 or less, or about 3.0 or less, or about 2.5 or less. For example, an acid solution (such as HCl solution) can be added to an aqueous suspension of the collagen IV-derived peptide (with or without other excipients) with high shear mixing to produce the collagen IV-derived peptide solution. For example, the HCl can be added slowly while mixing at 2000 to 20,000 rpm. Stirring can continue until the peptide is in solution, which can take, for example, one or several minutes. The solution can be filter sterilized, such as with a hydrophilic PVDF membrane (0.22 μm) or alternatively a nylon sterilizing filter.

In some embodiments, the collagen IV-derived peptide solution is mixed with a sodium chloride solution to prepare a final solution of 0.9% sodium chloride. In some embodiments, the sodium chloride solution is 1.8% sodium chloride, when used in equal volume to the collagen IV-derived peptide solution. The sodium chloride solution will further comprise sodium hydroxide to achieve the resulting pH of the suspension (pH of 6.0 to 8.0 or as already described). For example, the sodium chloride solution can contain 5 mM NaOH when used in equal volumes with the peptide solution. The sodium chloride solution may further comprise hyaluronic acid (or derivative) and/or cellulose or derivative (such as carboxymethylcellulose), as described. In various embodiments, the solution of the collagen IV-derived peptide and the sodium chloride solution are mixed for at least 4 hours, or at least 8 hours, or at least 10 hours, or at least about 15 hours to prepare the microparticle or nanoparticle suspension. The solutions can be mixed with stirring, for example at 500 to 2000 rpm (e.g., about 1200 rpm). In some embodiments, the mixing is from about 4 to about 24 hours, such as from 4 to about 18 hours, or from 4 to about 12 hours, or from 4 to about 10 hours. In some embodiments, the solution is mixed for about 18 hours, or more in some embodiments.

Alternatively or in addition, the solution of the collagen IV-derived peptide and the sodium chloride solution can be sonicated to prepare the microparticle or nanoparticle suspension, including to achieve the desired size (as already described).

Where the suspension is desired to include an additional agent (either therapeutic agent or imaging agent), the solution of the collagen IV-derived peptide or the sodium chloride solution comprises the additional agent.

In another aspect, the present disclosure provides alternative methods for making the suspension. Such methods include making the microparticle or nanoparticle suspension by a method comprising preparing a collagen IV-derived peptide gel, followed by homogenization to prepare the microparticle or nanoparticle suspension. For example, the collagen IV-derived peptide gel can be prepared by adding a solution of the collagen IV-derived peptide (including as described) to a mock vitreous comprising sodium hyaluronate and physiological pH (e.g., about 7.4) and physiological salt (equal to about 0.9% sodium chloride). Other features of the microparticle or nanoparticle suspension can be as described herein. Homogenization of the gel can be by any means, including by grinding, shearing, sonicating, and the like.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

As used herein, unless the context requires otherwise, the term “about” means±10% of an associated numerical value.

The invention will further be described in accordance with the following non-limiting examples.

### EXAMPLES

### Example 1: Microparticle and Nanoparticle Suspension of AXT107

Aqueous solutions of AXT107 precipitate to form a gel-like substance when administered into vitreous from animals and people. See WO 2020/198481 and 2019/0225670, which are hereby incorporated by reference in their entireties. The gel is formed when an aqueous solution of AXT107 hits the physiological pH, salt, and the viscosity of the hyaluronic acid of the vitreous. While this gelling features provides the basis for efficiently delivering a depot of therapeutic peptide into the vitreous, it has been discovered that the properties of the gel formed in the vitreous have some undesirable properties. For example, the gel can potentially spread into different parts of the vitreous resulting in floaters and fragments that can break off and move to the anterior chamber and cause an increase in intraocular pressure. Further, the properties of the vitreous can affect the physical properties of the gel, making it difficult to control the pharmacodynamics with intravitreal injection. For example, vitreous can be heterogeneous with distinct liquid and more solid regions, the vitreous between patients can also be different, and the effect of diseases such as diabetic macular edema and age-related macular degeneration on the vitreous is incompletely understood. These factors make intravitreal administration of the aqueous AXT107 solution not therapeutically desirable.

The following examples demonstrate microparticulate and nanoparticulate suspensions of the AXT107 peptide that provide for better control of AXT107 delivery. The particles in these suspensions are of defined size and reproducible. In addition, the suspensions can be administered into the suprachoroidal space as an alternative to intravitreal administration.

The following examples demonstrate production of an AXT107 peptide suspension. The peptide suspension is based on the mixture of two sterile-filtered solutions, an active pharmaceutical ingredient (API) solution and a suspending solution, followed by a long mixing period (e.g., approximately 18 hours, e.g., using a stir bar) and subsequent filling. The two solutions can be prepared at two-fold final concentrations to allow for final concentrations upon mixing with equal volumes of both solutions. Exemplary AXT107 suspensions contain 5 mg/mL to 10 mg/ml API, 5% sucrose, and 0.9% sodium chloride (154 mM). In other iterations, the suspension can contain other ingredients, such as sodium hyaluronate or sodium carboxymethyl cellulose, which can be included in the suspending solution for example.

To generate a sterile API solution, a powder containing the AXT107 peptide was transferred to a mixing vessel containing water and sucrose and stirred with a stir bar at 1200 rpm on a stir plate. Once the API appeared evenly suspended, HCl (to 5 mM) was added slowly to the stirring mixture. After all the HCl was added, the solution had a slightly more clarified appearance relative to the cloudiness of the initial suspension. The solution was then further processed by high shear mixing at 16000 rpm for 1 minute using a 5 mm probe. This step could alternatively be conducted with larger scale high shear mixers that typically operate under 5000 RPM. The fully dissolved solution appears relatively clear with some opalescence. This resulting solution is then sterilized by filtration through a 0.22 μm nylon sterilizing filter. The solutions may also be filtered through a hydrophilic PVDF membrane. When combined with sodium chloride as an excipient, the AXT107 peptide forms a microparticle suspension. Alternatively, sonication instead of mixing can be used to prepare the API solution.

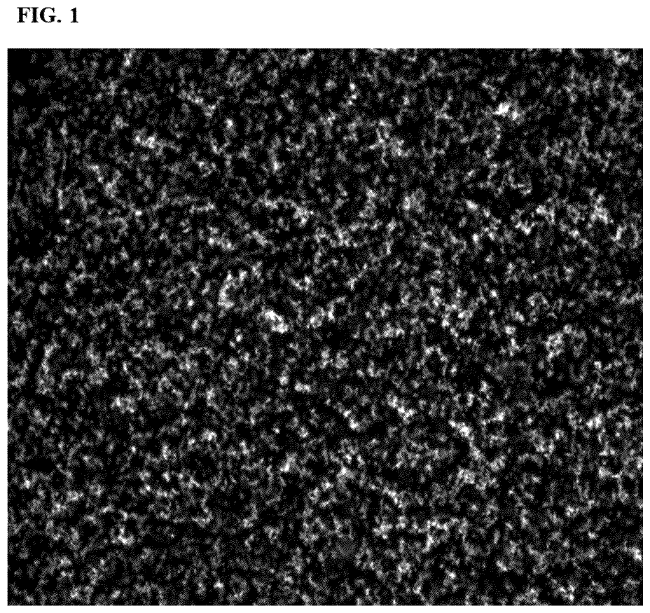

In certain experiments, a 10 mg/mL of solution containing the AXT107 peptide in 10% sucrose, water, and 5 mM HCl was generated using a high shear mixer. An equal volume of a separate suspending solution containing 1.8% NaCl and 5 mM NaOH was added to the solution containing the AXT107 peptide solution to precipitate the AXT107 peptide. Microparticles of between about 5 to 15 microns are generated by stirring the mixture of the two solutions for between 4 to 18 hours at 1200 rpm. This process results in a 5 mg/ml suspension of AXT107 peptide containing microparticles, 5% sucrose, and 0.9% NaCl. A 10 mg/mL microparticle suspension containing the AXT107 peptide is generated by starting with a 20 mg/mL solution of the AXT107 peptide and keeping the rest of the process the same. DAPI, a dye that is known to fluoresce upon binding to DNA, appears to also bind AXT107 particles and fluoresces because of the binding. When the mixture of DAPI and AXT107 peptide suspension is added to a hemocytometer and observed by fluorescence microscopy, the size of the particles can be estimated by comparing the dimensions of the grid on the hemocytometer. FIG. 1 shows a fluorescent microscopic image of an exemplary microparticle suspension of AXT107 peptide.

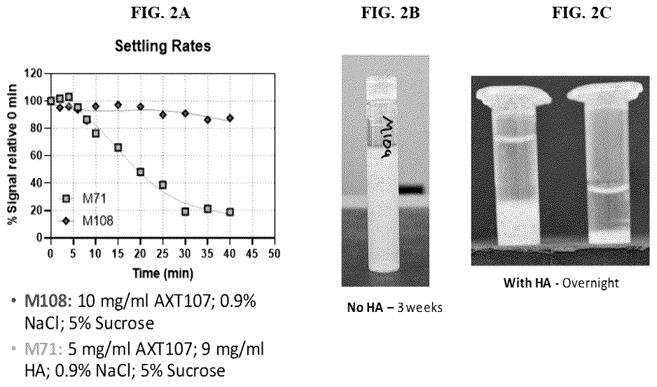

AXT107 suspensions were evaluated to determine the rate at which the suspensions settle. The rate at which a suspension settles can be an important factor in manufacturing. For example, while the suspension is being stirred, the particles remain in suspension, but particles may settle when they move through the tubing from the container in which they are being stirred to the vials to which they are being added. A suspension that settles slowly is preferable to one that settles quickly to avoid settling of the particles in the tubing. The settling rate of a suspension developed with NaCl in the suspending solution was compared with a suspension that included both NaCl and hyaluronic acid (HA) (4000 mol. wt.) in the suspending solution. As seen in FIG. 2A and FIG. 2B, the suspension that does not contain HA settles slowly compared to the solution with HA (FIGS. 2A and 2C). FIG. 2B shows an image of a suspension containing NaCl, but no hyaluronic acid. The suspension did not settle when kept at room temperature for 3 weeks. FIG. 2C shows that a suspension containing HA in addition to NaCl settles when placed at room temperature overnight.

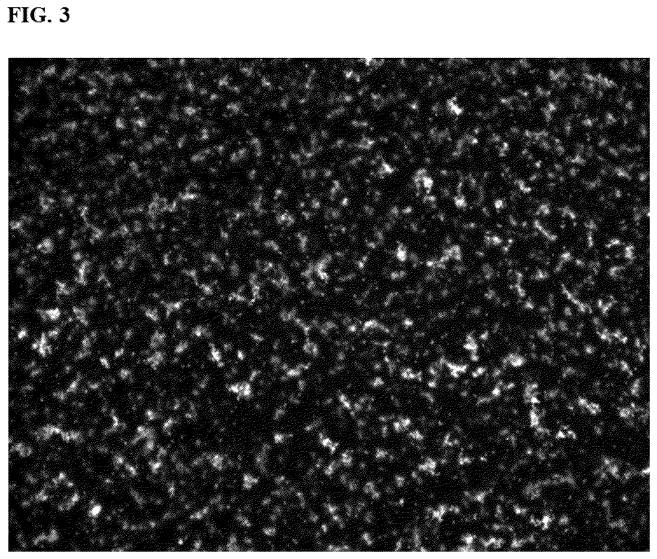

Additional experiments were conducted to evaluate a microparticle suspension of AXT107 peptide prepared by precipitating the AXT107 peptide with NaHA and NaCl (FIG. 3). In these experiments, when both sodium hyaluronate (4000 mol. wt.) and sodium chloride are included in the suspending solution, the AXT107 peptide forms a microparticle suspension. There can be advantages to including HA to develop microparticle suspensions. The HA containing particles can release the AXT107 peptide more slowly, thus potentially providing a longer duration product. An image of the particles visualized by fluorescence microscopy after adding DAPI is shown in FIG. 3. These particles are also in the size range of 5-15 microns.

Additional experiments demonstrated a nanoparticle suspension of AXT107 peptide prepared by precipitating the AXT107 peptide with NaHA, NaCMC (sodium carboxymethylcellulose), and NaCl. If NaCMC is used in addition to NaHA and NaCl in the suspending solution, and the precipitate is sonicated instead of stirred, a nanoparticle suspension is generated. Fluorescence microscopy with DAPI shows a faint glow instead of discrete particles. The nanoparticle nature of the particles is confirmed by dynamic light scattering (DLS). A suspension comprising nanoparticles can potentially release faster than microparticles suspensions. An exemplary suspension according to this example was prepared with 5 mg/mL AXT107, 9 mg/mL HA (4000 mol. wt.), 0.5% CMC, 5% sucrose, and 0.9% NaCl.

In alternative embodiments, microparticles can be prepared and fragmented. For example, a vitreous-like solution (“mock vitreous”) was prepared with sodium hyaluronate and used for in vitro pre-fragmented gel formation experiments. The AXT107 peptide solution is added to the mock vitreous, upon which the peptide forms a gel. These gels can be homogenized after formation to break down both peptide self-assembly and peptide/hyaluronate co-assembly, generating suspended microparticles comparable to those formed in excipient formulation with mixing.

The results of these experiments show the development of microparticle suspensions and nanoparticle suspensions of the AXT107 peptide. The particles in these suspensions are of defined size and are reproducible.

### Example 2: Incorporation of Therapeutic Cargos into Suspensions

The following experiments demonstrate incorporation of protein cargos with the AXT107 peptide microparticles. The AXT107 peptide, as well as other analogous peptide sequences, exhibits the capability to incorporate proteins within the microparticles in suspension. Currently, therapeutic proteins or antibodies injected into the vitreous must be administered between once every month to once every three months. The frequency of treatment is a large burden to patients and often leads to poor compliance, as well as to safety issues. Therapeutic proteins (e.g., antibodies, small molecules) incorporated into a collagen IV derived peptide microparticle suspension can be released in a sustained manner over many months, thus allowing for much less frequent dosing. In these experiments, a proteinaceous cargo that is encapsulated interacts with the peptide microparticles, thereby slowing down the release of the collagen IV derived peptide. As a result, large therapeutic proteins, as well as therapeutic non-proteinaceous small molecules, such as kinase inhibitors and aptamers that are conjugated to small peptides, can be delivered in a sustained manner to the back of the eye.

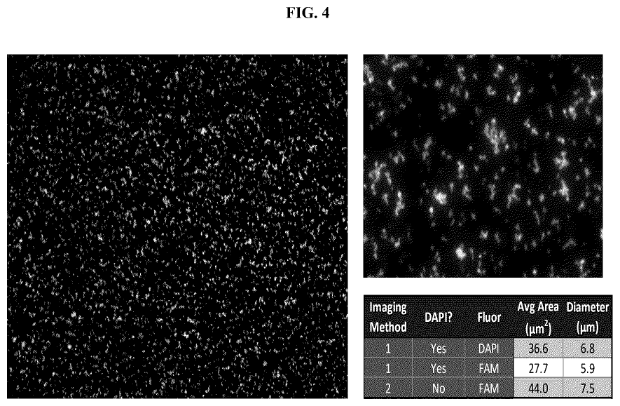

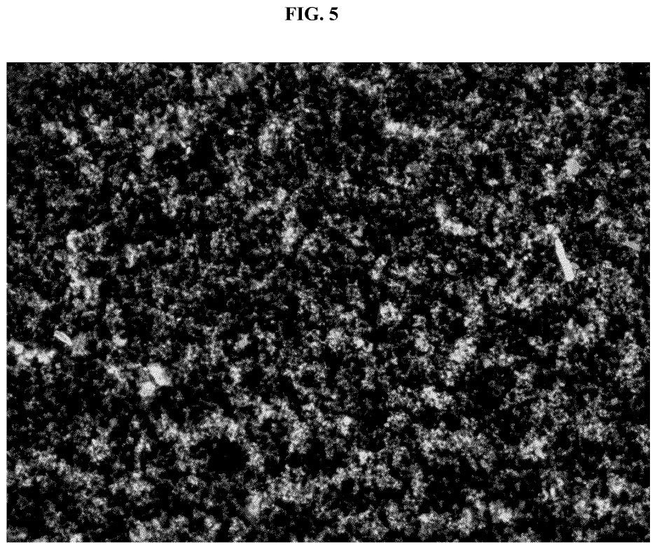

The following example demonstrates that AXT107 peptide microparticles can incorporate FITC-labeled bovine serum albumin (BSA) and FAM-AXT107 to form a suspension (FIG. 4). The microparticles containing the AXT107 peptide were developed by first dissolving the unlabeled AXT107 with either FITC-BSA or FAM-AXT107. The solutions were then precipitated by adding a suspending solution containing HA and NaCl. DAPI was then added to visualize the AXT107 peptide microparticles. The fluorescence signal from FITC-BSA or FAM-AXT107 was found to overlap with the DAPI signal confirming that the AXT107 peptide and the labeled BSA, or the labeled AXT107 peptide, were present in the same microparticles (FIG. 5).

These examples demonstrate that collagen IV-derived biomimetic peptide-based microparticle suspensions can be used incorporate therapeutic cargo molecules (e.g., antibodies, small molecules), and may provide for a release of active agent over a long duration of time with administration of a small volume.

### Example 3: Biological Activity of AXT107 Suspensions

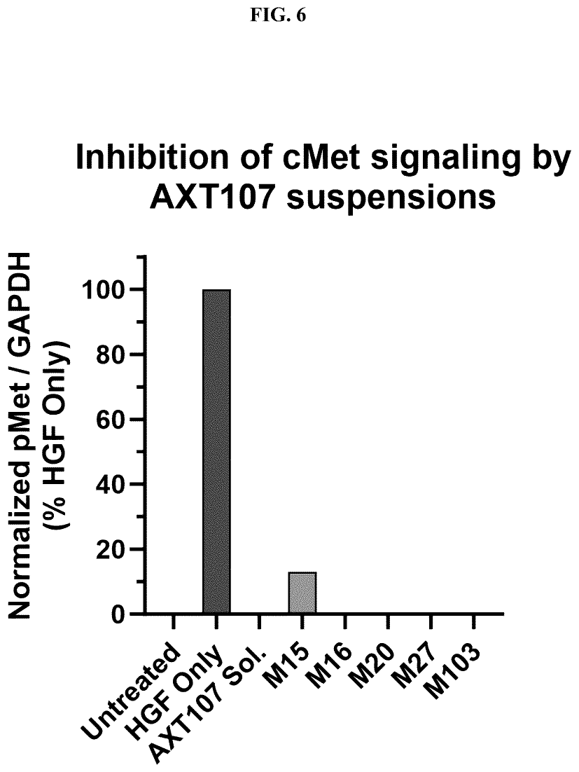

The biological activity of various suspension formulations were evaluated. In these experiments, endothelial cells (HUVEC cells) were induced with hepatocyte growth factor (HGF), leading to c-Met phosphorylation. When the cells were pre-treated with several of the suspension formulations at a concentration of 100 μM for 90 min, prior to addition of HGF, c-Met phosphorylation was found to be inhibited (FIG. 6). c-Met is one of many receptor tyrosine kinases that are involved in neovascularization, and is used as a surrogate for all of the receptors in these experiments because it is robustly phosphorylated when endothelial cells are treated with HGF. The assessed formulations were generated by mixing solutions of AXT107 in sucrose with varying suspending reagents to induce peptide precipitates. These precipitates were then disrupted into particles by sonication, except for M27 and M103, which were disrupted by stirring at 1200 rpm for 18 hours. All suspensions contained final concentrations of 5 mg/ml AXT107 and 5% sucrose. Suspending reagents included 9 mg/ml HA for M15, 9 mg/ml HA and 0.9% NaCl for M16, 9 mg/ml HA, 0.5% CMC, and 0.9% NaCl for M20, 9 mg/ml HA and 0.9% NaCl for M27, and 0.9% NaCl for M103. For treatment, these suspensions were diluted in HUVEC cultures to 100 μM AXT107.

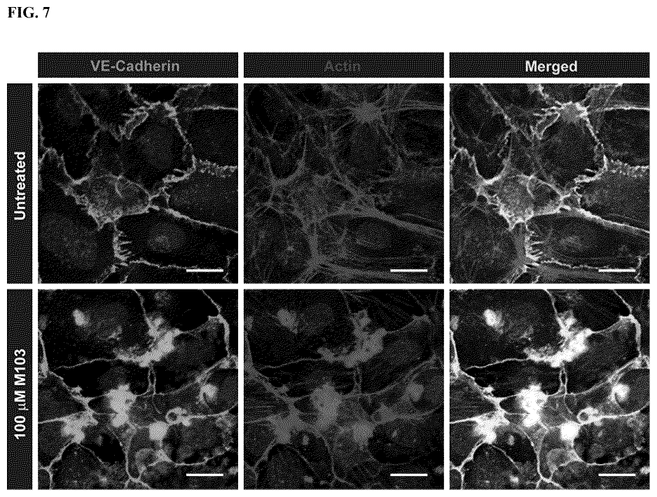

FIG. 7 shows a fluorescent microscopic image of endothelial cells (HUVEC) untreated (top panels) or treated with the AXT107 microparticulate suspension M103 (AXT107 in 5% sucrose precipitated by 0.9% sodium chloride and disrupted into particles by stir bar) (bottom panels). Cells were stained using antibodies against the cell-junction marker VE-cadherin, actin, and nucleus marker DAPI. Scale bar indicates 25 μm. These data demonstrate that treatment of endothelial cells with AXT107 microparticle suspension stabilizes cell-cell junctions by redistributing actin to the periphery of the cell, rather than across the cell, and creates a smooth VE-cadherin distribution, rather than the more jagged appearance in the untreated cells. This suggests a more continuous cell-cell boundary rather than a more permeable one.

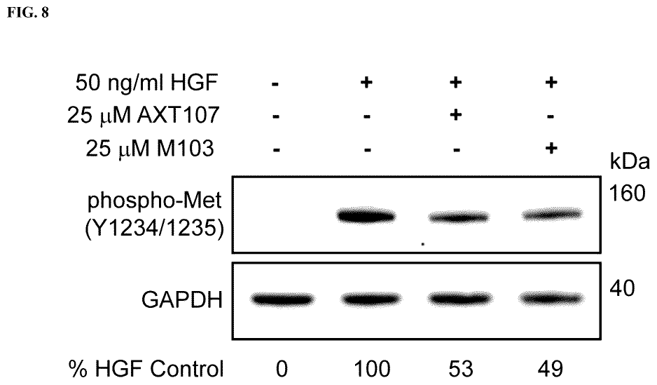

FIG. 8 is a Western blot image comparing inhibition of cMet phosphorylation by AXT107 solution and M103 suspension (AXT107 in 5% sucrose precipitated by 0.9% sodium chloride and disrupted into microparticles by stir bar). Endothelial cells (HUVEC) were treated with 25 μM AXT107 solution or M103, and inhibition of c-Met phosphorylation induced by hepatocyte growth factor (HGF ((50 ng/mL) determined. As shown, AXT107 solution and M103 suspension show similar inhibition of cMet phosphorylation.

### Example 4: Pharmacokinetic and Tolerability Evaluation of AXT107 Peptide Administered by Suprachoroidal Injection

This example evaluates the pharmacokinetics (PK)/tolerability of the AXT107 suspension (5% sucrose, 0.9% NaCl) at 4 different dose levels administered by suprachoroidal (Sch) injection in female Dutch Belted rabbits. In Phase 1 of these experiments, animals received a 100 μL SCh injection in both eyes on Day 0. In these experiments, Group 1 received 469 μg of AXT107 peptide, Group 2 received 246 μg of AXT107 peptide, and Group 3 received vehicle (i.e., 5% sucrose in saline).

Ocular examinations were performed at baseline, and on Days 1, 3, 7, 15, 30, 60, and 90. On Day 1, all eyes had inflammation, generally mild. At all other post-dose OE timepoints, inflammation was rare and mild. Tonometry was performed at baseline, and on Days 1, 3, 7, 15, 30, 60, and 90. At all timepoints, the intraocular pressure (IOP) of all groups remained within the normal range for this strain and species. Optical coherence tomography (OCT) was performed at baseline, post-dose to confirm SCh dosing, and on Day 15. The total retina thickness (TRT) in all groups was generally close to baseline, with the exception of a slight decrease in the TRT in Group 2 on Day 0. The suprachoroidal space (SCS) was similar in Groups 1 and 2, and decreased in Group 3 at the Day 0 timepoint; the SCS measurements were 0 in all eyes at the Day 15 timepoint.

Blood was collected from Group 3 pre-dose, and in terminal animals on Day 90, and was processed for plasma. All eyes in Groups 1 and 2 and two Group 3 eyes were collected and dissected. Blood and ocular tissues underwent PK analysis. From Group 3, two eyes were collected and processed for hematoxylin & eosin (H&E) histopathology. There were no histologic abnormalities observed in either eye.

In general, both doses of the AXT107 peptide were well tolerated in these experiments.

In the next studies, animals received a 100 μL SCh injection (of AXT107 suspension, 5% sucrose and 0.9% NaCl) in both eyes on Day 0. Group 1 animals received 340 μg of AXT107 peptide, Group 2 animals received 219 μg AXT107 peptide, Group 3 animals received 142 μg of AXT107 peptide, and Group 4 animals received 115 μg AXT107 peptide. Ocular examinations were performed at baseline and on Days 1, 9, 30, 60, and 90. On Day 1, almost all eyes had inflammation, generally mild. At all other post-dose timepoints, inflammation was rare and mild.

Tonometry was performed at baseline and on Days 1, 9, 30, 60, and 90. At all timepoints, the IOP of all groups remained within the normal range for this strain and species.

OCT was performed at baseline, post-dose to confirm SCh dosing, and in terminal animals on Days 15 and 30. The TRT of Groups 1 and 2 was decreased from baseline on Days 0, 15, and 30. The TRT of Groups 3 and 4 was near baseline on Days 0 and 30, and decreased on Day 15. On Day 0, the SCS was smallest in Group 1 eyes, similar in Group 2 and Group 3 eyes, and largest in Group 4 eyes; the SCS measurements were 0 in all eyes at all other timepoints.

On Days 1, 15, 30, 60, and 90, n=2 animals/group were euthanized and plasma and ocular tissues were collected for PK. On Day 90, an additional Group 1 animal was euthanized and OU were processed for H&E histopathology. Both eyes had mild mononuclear cell infiltrate in the limbal conjunctiva, which was considered to be an incidental finding due to needle damage and unrelated to the administration of the AXT107 peptide.

Plasma and ocular tissues collected during the study were analyzed for AXT107 peptide PK parameters by non-GLP liquid chromatography with tandem-mass spectrometry (LC/MS/MS). These experiments demonstrated that all doses of the AXT107 peptide were well tolerated and drug levels were present in tissues associated with ocular vascular diseases, such as choroid/RPE and retina, but absent in plasma or aqueous humor.

These experiments demonstrate that SCh injection of all doses of the AXT107 suspension was well tolerated, and drug levels were present in appreciable amounts in tissues associated with ocular vascular diseases, including choroid/RPE and sclera for the 90 day period, and retina for 30 days. Conversely, the AXT107 peptide was absent in plasma and aqueous humor at all timepoints.

1. A pharmaceutical composition comprising a microparticle or nanoparticle suspension of a collagen IV-derived peptide or pharmaceutically-acceptable salt thereof.

2. The pharmaceutical composition of claim 1, wherein the peptide comprises the amino acid sequence LRRFSTXPXXXXDINDVXNF (SEQ ID NO: 1), where X is a standard amino acid or a non-genetically-encoded amino acid.

3. The pharmaceutical composition of claim 1, wherein the peptide comprises the amino acid sequence LRRFSTXPXXXXNINNVXNF (SEQ ID NO: 2), where X is a standard amino acid or a non-genetically-encoded amino acid.

4. The pharmaceutical composition of claim 1, wherein the peptide comprises or consists of the amino acid sequence LRRFSTAPFAFIDINDVINF (SEQ ID NO: 3) or LRRFSTAPFAFININNVINF (SEQ ID NO: 4).

5. The pharmaceutical composition of any one of claims 1 to 4, wherein the concentration of the collagen IV-derived peptide is from about 1 mg/mL to about 15 mg/mL, or from about 4 mg/mL to about 12 mg/mL.

6. The pharmaceutical composition of claim 5, wherein the concentration of the collagen IV-derived peptide is about 5 mg/mL or about 10 mg/mL.

7. The pharmaceutical composition of any one of claims 1 to 6, wherein the composition comprises sodium chloride, which is optionally 0.9% sodium chloride.

8. The pharmaceutical composition of claim 7, wherein the composition comprises one or more tonicity adjusting mono- or disaccharides.

9. The pharmaceutical composition of claim 8, wherein the composition comprises sucrose, which is optionally about 5% sucrose.

10. The pharmaceutical composition of any one of claims 1 to 9, wherein the composition has a pH in the range of 6 to 8, and which is optionally about pH 7.0 or about 7.4.

11. The pharmaceutical composition of claim 10, wherein the composition consists essentially of, or consists of, the peptide of SEQ ID NO: 3, about 5% sucrose, and about 0.9% sodium chloride.

12. The pharmaceutical composition of any one of claims 1 to 10, wherein the composition further comprises hyaluronic acid or a hyaluronic acid derivative, or a pharmaceutically acceptable salt thereof.

13. The pharmaceutical composition of claim 12, wherein the composition comprises sodium hyaluronate, which is optionally a low molecular weight sodium hyaluronate.

14. The pharmaceutical composition of any one of claims 1 to 13, wherein the composition further comprises cellulose or a cellulose derivative, or pharmaceutically acceptable salt thereof, and which is optionally carboxymethylcellulose.

15. The pharmaceutical composition of any one of claims 1 to 14, wherein the suspension is a microparticle suspension where the majority of particles are in the range of about 1 to about 200 microns in size, or in the range of about 10 to about 100 microns in size.

16. The pharmaceutical composition of any one of claims 1 to 11, wherein the suspension is a nanoparticle suspension.

17. The pharmaceutical composition of claim 16, wherein the composition comprises sodium hyaluronate which is optionally a low molecular weight sodium hyaluronate and carboxymethylcellulose.

18. The pharmaceutical composition of any one of claims 1 to 17, wherein the composition comprises an additional therapeutic agent or imaging agent or label, which is optionally conjugated to the collagen IV-derived peptide.

19. The pharmaceutical composition of claim 18, wherein the therapeutic agent is a protein, peptide, small molecule, polymer, polynucleotide, oligonucleotide, aptamer, carbohydrate, or lipid.

20. The pharmaceutical composition of claim 19, wherein the therapeutic agent is a protein, and the protein is optionally an antibody, enzyme, cytokine, or soluble receptor ligand.

21. The pharmaceutical composition of claim 19, wherein the therapeutic agent is selected from Ranibizumab, Aflibercept, GSK933776, THR-317, RO6867461, REGN910, DS7080a, TK001, Brolucizumab, REGN2176-3, and iSONEP.

22. The pharmaceutical composition of claim 19, wherein the therapeutic agent targets a protein of the complement pathway, optionally wherein the complement pathway target is complement factor 1 (C1), C1s, C1q, C1r, complement factor 2 (C2), complement factor 3 (C3), C3a, C3b, C3bBb, complement factor 4 (C4), C4b2b complement factor 5 (C5), C5a, C5b, C5b-9, factor properdin, complement factor B, complement factor D.

23. The pharmaceutical composition of claim 19, wherein the therapeutic agent is complement factor H or complement factor I, or a mimetic thereof.

24. The pharmaceutical composition of claim 19, wherein the therapeutic agent is a kinase inhibitor or corticosteroid.

25. A method for treating an ocular condition characterized by inflammation and/or neovascularization, comprising, administering an effective amount of the pharmaceutical composition of any one of claims 1 to 24 to a subject in need thereof.

26. The method of claim 25, wherein the ocular condition is selected from age-related macular degeneration (AMD), diabetic macular edema (DME), diabetic retinopathy, macular edema (ME), neovascular glaucoma, and retinopathy of prematurity.

27. The method of claim 26, wherein the subject has a condition selected from dry or wet AMD.

28. The method of claim 27, wherein the subject has dry AMD and geographic atrophy.

29. The method of claim 25, wherein the subject has uveitis.

30. The method of claim 25, wherein the ocular condition includes inflammation associated with an autoimmune condition.

31. The method of claim 25, wherein the ocular condition is a proliferative condition, optionally selected from retinoblastoma, ocular rhabdomyosarcoma, choroidal melanoma, and uveal melanoma.

32. The method of any one of claims 25 to 31, wherein the pharmaceutical composition is administered by suprachoroidal injection.

33. The method of any one of claims 25 to 31, wherein the pharmaceutical composition is administered by intravitreal injection.

34. The method of any one of claims 25 to 33, wherein the administration is no more frequent than once every other month.

35. The method of claim 34, wherein the injection is no more frequent than once every three months, or no more frequent than once every four months.

36. The method of claim 34, wherein the administration is no more frequent than once every six months, or no more frequent than once every year.

37. The method of claim 34, wherein the administration is about once every six months.

38. The method of any one of claims 25 to 37, wherein the composition is administered at a volume of less than about 100 μL or less than about 50 μL or less than about 25 μL.

39. A method for treating an ocular condition characterized by inflammation and/or neovascularization, comprising, administering an effective amount of a pharmaceutical composition comprising a collagen IV-derived peptide by suprachoroidal administration.

40. The method of claim 39, wherein the composition is a microparticle or nanoparticle suspension of any one of claims 1 to 22.

41. The method of claim 39 or 40, wherein the ocular condition is selected from age-related macular degeneration (AMD), diabetic macular edema (DME), diabetic retinopathy, macular edema (ME), neovascular glaucoma, and retinopathy of prematurity.

42. The method of claim 41, wherein the subject has a condition selected from dry or wet AMD.

43. The method of claim 42, wherein the subject has dry AMD and geographic atrophy.

44. The method of claim 39, wherein the subject has uveitis.

45. The method of claim 39, wherein the ocular condition includes inflammation associated with an autoimmune condition.

46. The method of claim 39, wherein the ocular condition is a proliferative condition, optionally selected from retinoblastoma, rhabdomyosarcoma, choroidal melanoma, and uveal melanoma.

47. The method of any one of claims 39 to 46, wherein the administration is no more frequent than once every other month.

48. The method of claim 47, wherein the injection is no more frequent than once every three months, or no more frequent than once every four months.

49. The method of claim 47, wherein the administration is no more frequent than once every six months, or no more frequent than once every year.

50. The method of claim 47, wherein the administration is about once every six months.

51. The method of any one of claims 39 to 50, wherein the composition is administered at a volume of less than about 100 μL or less than about 50 μL or less than about 25 μL.

52. A method for making a microparticle or nanoparticle suspension of a collagen IV-derived peptide comprising:
preparing a solution of the collagen IV-derived peptide,
mixing the solution of the collagen IV-derived peptide with a physiological salt solution, to prepare the microparticle or nanoparticle suspension.

53. The method of claim 52, wherein the collagen IV-derived peptide comprises the amino acid sequence LRRFSTXPXXXXNINNVXNF (SEQ ID NO: 1) or LRRFSTXPXXXXDINDVXNF (SEQ ID NO: 2), where X is a standard amino acid or a non-genetically-encoded amino acid.

54. The method of claim 53, wherein the peptide comprises or consists of the amino acid sequence LRRFSTAPFAFIDINDVINF (SEQ ID NO:3) or LRRFSTAPFAFININNVINF (SEQ ID NO: 4).

55. The method of any one of claims 52 to 54, wherein the concentration of the collagen IV-derived peptide in the suspension is from about 1 mg/mL to about 12 mg/mL, or from about 5 mg/mL to about 10 mg/mL.

56. The method of claim 55, wherein the concentration of the peptide in the solution of the collagen IV-derived peptide is from about 2 mg/mL to about 24 mg/mL.

57. The method of claim 54 or 55, wherein the collagen IV-derived peptide solution comprises one or more tonicity adjusting mono- or disaccharides.

58. The method of claim 57, wherein the collagen IV-derived peptide solution comprises sucrose, which is optionally about 10% sucrose.

59. The method of any one of claims 52 to 58, wherein the collagen IV-derived peptide solution further comprises one or more cryoprotectants, and where the cryoprotectant is optionally DMSO.

60. The method of any one of claims 52 to 59, wherein the collagen IV-derived peptide solution has a pH of less than 5.0, or about 4.0 or less, or about 3.0 or less, or about 2.5 or less.

61. The method of claim 60, wherein HCl is added to an aqueous suspension of the collagen IV-derived peptide with high shear mixing to produce the collagen IV-derived peptide solution.

62. The method of claim 61, wherein the collagen IV-derived peptide solution is added to a sodium chloride solution to prepare a final solution of 0.9% sodium chloride.

63. The method of claim 62, wherein the sodium chloride solution is 1.8% sodium chloride.

64. The method of any one of claims 52 to 63, wherein the solution of the collagen IV-derived peptide and the sodium chloride solution are mixed for at least 4 hours, or at least 8 hours, or at least 10 hours, to prepare the microparticle or nanoparticle suspension.

65. The method of any one of claims 52 to 64, wherein the wherein the solution of the collagen IV-derived peptide and the sodium chloride solution are sonicated to prepare the microparticle or nanoparticle suspension.

66. The method of any one of claims 52 to 65, wherein the composition has a pH in the range of 6 to 8, and which is optionally about pH 7.0 or about pH 7.4.

67. The method of any one of claims 52 to 66, wherein the sodium chloride solution further comprises hyaluronic acid or derivative thereof or pharmaceutically acceptable salt thereof.

68. The method of any one of claims 52 to 67, wherein the sodium chloride solution further comprises cellulose or a cellulose derivative, and which is optionally carboxymethylcellulose.

69. The method of any one of claims 52 to 68, wherein the suspension is a microparticle suspension with a mean particle size in the range of about 1 to about 200 microns, or in the range of about 10 to about 100 microns.

70. The method of any one of claims 52 to 69, wherein the suspension is a nanoparticle suspension.

71. The method of claim 70, wherein the sodium chloride solution comprises sodium hyaluronate and carboxymethylcellulose.

72. The method of any one of claims 52 to 71, wherein the solution of the collagen IV-derived peptide or the sodium chloride solution comprises an additional therapeutic agent or imaging agent or label.

73. A method for making a microparticle or nanoparticle suspension of a collagen IV-derived peptide comprising:
preparing a collagen IV-derived peptide gel, followed by homogenization to prepare the microparticle or nanoparticle suspension.

74. The method of claim 73, wherein the collagen IV-derived peptide gel is prepared by adding a solution of the collagen IV-derived peptide to a mock vitreous comprising sodium hyaluronate.

75. The method of claim 73 or 74, wherein the microparticle or nanoparticle suspension is according to any one of claims 1 to 24.


SEQUENCE LISTING (36 sequences)

SEQ ID NO: 1 - protein, 20 aa, synthetic construct
  VARIANT 7: note = X is a standard amino acid or a non-genetically-encoded amino acid
  VARIANT 9: note = X is a standard amino acid or a non-genetically-encoded amino acid
  VARIANT 10: note = X is a standard amino acid or a non-genetically-encoded amino acid
  VARIANT 11: note = X is a standard amino acid or a non-genetically-encoded amino acid
  VARIANT 12: note = X is a standard amino acid or a non-genetically-encoded amino acid
  VARIANT 18: note = X is a standard amino acid or a non-genetically-encoded amino acid
LRRFSTXPXXXXDINDVXNF

SEQ ID NO: 2 - protein, 20 aa, synthetic construct
  VARIANT 7: note = X is a standard amino acid or a non-genetically-encoded amino acid
  VARIANT 9: note = X is a standard amino acid or a non-genetically-encoded amino acid
  VARIANT 10: note = X is a standard amino acid or a non-genetically-encoded amino acid
  VARIANT 11: note = X is a standard amino acid or a non-genetically-encoded amino acid
  VARIANT 12: note = X is a standard amino acid or a non-genetically-encoded amino acid
  VARIANT 18: note = X is a standard amino acid or a non-genetically-encoded amino acid
LRRFSTXPXXXXNINNVXNF

SEQ ID NO: 3 - protein, 20 aa, synthetic construct
LRRFSTAPFAFIDINDVINF

SEQ ID NO: 4 - protein, 20 aa, synthetic construct
LRRFSTAPFAFININNVINF

SEQ ID NO: 5 - protein, 18 aa, synthetic construct
LRRFSTMPFMFNINNVNF

SEQ ID NO: 6 - protein, 18 aa, synthetic construct
LRRFSTMPAMFNINNVNF

SEQ ID NO: 7 - protein, 18 aa, synthetic construct
LRRFSTMPFAFNINNVNF

SEQ ID NO: 8 - protein, 18 aa, synthetic construct
LRRFSTMPFMANINNVNF

SEQ ID NO: 9 - protein, 17 aa, synthetic construct
LRRFSTMPFFNINNVNF

SEQ ID NO: 10 - protein, 17 aa, synthetic construct
LRRFSTMPFMNINNVNF

SEQ ID NO: 11 - protein, 20 aa, synthetic construct
LRRFSTMPFMFSNINNVSNF

SEQ ID NO: 12 - protein, 20 aa, synthetic construct
LRRFSTMPFMFANINNVANF

SEQ ID NO: 13 - protein, 20 aa, synthetic construct
LRRFSTMPFMFININNVINF

SEQ ID NO: 14 - protein, 20 aa, synthetic construct
LRRFSTMPFMFTNINNVTNF

SEQ ID NO: 15 - protein, 18 aa, synthetic construct
LRRFSTMPFMFNINNVNF

SEQ ID NO: 16 - protein, 20 aa, synthetic construct
LRRFSTMPFMFVNINNVVNF

SEQ ID NO: 17 - protein, 20 aa, synthetic construct
LRRFSTMPFAFININNVINF

SEQ ID NO: 18 - protein, 20 aa, synthetic construct
LRRFSTMPFAFININNVINF

SEQ ID NO: 19 - protein, 20 aa, synthetic construct
LRRFSTAPFAFININNVINF

SEQ ID NO: 20 - protein, 21 aa, synthetic construct
LRRFSTAPFDAFIDINDVINF

SEQ ID NO: 21 - protein, 20 aa, synthetic construct
LRRFSTAPFAFIDINDVINW

SEQ ID NO: 22 - protein, 23 aa, synthetic construct
LRRLRRFSTAPFAFIDINDVINF

SEQ ID NO: 23 - protein, 20 aa, synthetic construct
LRRFSTAPFAFIDINDVINF

SEQ ID NO: 24 - protein, 20 aa, synthetic construct
LRRFSTAPFAFIDINDVINF

SEQ ID NO: 25 - protein, 7 aa, synthetic construct
FNINNVN

SEQ ID NO: 26 - protein, 9 aa, synthetic construct
FTNINNVTN

SEQ ID NO: 27 - protein, 10 aa, synthetic construct
FININNVINF

SEQ ID NO: 28 - protein, 10 aa, synthetic construct
FSNINNVSNF

SEQ ID NO: 29 - protein, 10 aa, synthetic construct
FANINNVANF

SEQ ID NO: 30 - protein, 8 aa, synthetic construct
FNINNVNF

SEQ ID NO: 31 - protein, 10 aa, synthetic construct
FVNINNVVNF

SEQ ID NO: 32 - protein, 10 aa, synthetic construct
FIDINDVINF

SEQ ID NO: 33 - protein, 10 aa, synthetic construct
FIDINDVINW

SEQ ID NO: 34 - protein, 9 aa, synthetic construct
FTDINDVTN

SEQ ID NO: 35 - protein, 8 aa, synthetic construct
ANINNVNF

SEQ ID NO: 36 - protein, 7 aa, synthetic construct
NINNVNF